(19) United States
(12) Patent Application Publication
(10) Pub. No.: US 2010/0022402 A1
(43) Pub. Date: Jan. 28, 2010

(54) Methods and Compositions for the In Vitro High-Throughput Detection of Protein/Protein Interactions

(76) Inventors: Sancar Adhya, Gaithersburg, MD (US); Amos Oppenheim, Jerusalem (IL)

Correspondence Address: NATIONAL INSTITUTES OF HEALTH, P. O. BOX 2903, MINNEAPOLIS, MN, 55402, US

(21) Appl. No.: 11/719,925

(22) PCT Filed: Nov. 22, 2005
(86) PCT No.: PCT/US05/42612

§ 371 Date: Jun. 22, 2009

Related U.S. Application Data
(60) Provisional application No. 60/629,933, filed on Nov. 23, 2004.

(51) Int. Cl. C40B 30/04; C12Q 1/70

(52) U.S. Cl. 506/9; 435/5

(57) ABSTRACT

The present invention relates to methods and compositions for the identification and/or assessment of protein/protein interactions, and in particular to methods and compositions for accomplishing the high-throughput detection of interactions of proteins displayed on the surfaces of lambdoid bacteriophage particles.

### CROSS-REFERENCE TO RELATED APPLICATIONS

This application claims priority to U.S. Patent Application Ser. No. 60/629,933 (filed on Nov. 23, 2004), which application is herein incorporated by reference in its entirety.

### FIELD OF THE INVENTION

The present invention relates to methods and compositions for the identification and/or assessment of protein/protein interactions, and in particular to methods and compositions for accomplishing the high-throughput detection of interactions of proteins displayed on the surfaces of lambdoid bacteriophage particles.

### BACKGROUND OF THE INVENTION

One of the central challenges in proteomics involves defining interacting epitopes of multi-complex protein structures in normal or diseased cells. The ability to address this challenge is complicated by the limited number of high-throughput systems that may be used to detect or identify protein/protein interactions.

One widely employed high throughput system is the yeast two-hybrid system (Fields, S. et al. (1989) “A NOVEL GENETIC SYSTEM TO DETECT PROTEIN-PROTEIN INTERACTIONS,” Nature 340:245-246; Fields, S. et al.; U.S. Pat. No. 5,283,173). The yeast two-hybrid system utilizes the reconstitution of a transcriptional activator like GAL4 (Johnston, M. (1987) “A MODEL FUNGAL GENE REGULATORY MECHANISM: THE GAL GENES OF Saccharomyces Cerevisiae, Microbiol. Rev. 51:458-476) through the interaction of two test protein domains that are part of fusion proteins with two functional units of the transcriptional activator. The reconstitution of the transcriptional activator is monitored by the activation of a reporter gene, such as the lacZ gene, that is under the influence of a promoter that contains a binding site (referred to as an “Upstream Activating Sequence” or “UAS”) for the DNA-binding domain of the transcriptional activator. The method is most commonly used to detect an interaction between two known proteins (Fields, S. et al. (1989) “A NOVEL GENETIC SYSTEM TO DETECT PROTEIN-PROTEIN INTERACTIONS,” Nature 340:245-246) and to identify interacting proteins from a population that would bind to a known protein (Durfee et al. (1993) “THE RETINOBLASTOMA PROTEIN ASSOCIATES WITH THE PROTEIN PHOSPHATASE TYPE 1 CATALYTIC SUBUNIT,” Genes Dev. 7:555-569; Gyuris et al. (1993) “CDI1, A HUMAN G1 AND S PHASE PROTEIN PHOSPHATASE THAT ASSOCIATES WITH CDK2,” Cell 75:791-803; Harper, J. W. et al. (1993) “THE P21 CDK-INTERACTING PROTEIN CIP1 IS A POTENT INHIBITOR OF G1 CYCLIN-DEPENDENT KINASES,” Cell 75:805-816; Vojtek, A. B. et al. (1993) “MAMMALIAN RAS INTERACTS DIRECTLY WITH THE SERINE/THREONINE KINASE RAF,” Cell 74:205-214). Two disadvantages of the yeast two-hybrid system are that the system tends to produce a high number of false positives and that it is not possible to manipulate the conditions under which protein/protein interactions are selected for because the protein/protein interactions occur within the yeast nucleus.

“Phage display systems” have been employed to detect and assess protein/protein interactions. In such systems, a test protein of a putative binding pair is expressed on the surface of bacteriophage particles. Phage display technology, which started with the identification of peptide epitopes recognized by monoclonal antibodies (Scott, J. K. et al. (1990) “SEARCHING FOR PEPTIDE LIGANDS WITH AN EPITOPE LIBRARY,” Science 249(4967):386-390), has grown into an approach for cloning human antibodies, studying ligand-receptor interactions, elucidating signal transduction pathways, delineating contact residues in interacting proteins, and isolating peptide inhibitors (Zozulya, S. et al. (1999) “MAPPING SIGNAL TRANSDUCTION PATHWAYS BY PHAGE DISPLAY,” Nat. Biotechnol. 17(12):1193-1198; Cortese, R. et al. (1996) “SELECTION OF BIOLOGICALLY ACTIVE PEPTIDES BY PHAGE DISPLAY OF RANDOM PEPTIDE LIBRARIES,” Curr Opin Biotechnol. 7(6):616-621; Rader, C. et al. (1997) “PHAGE DISPLAY OF COMBINATORIAL ANTIBODY LIBRARIES,” Curr Opin Biotechnol. 8(4):503-508.). Other applications of this technology include the production of gene/genome-fragment and cDNA libraries displaying, virtually every possible encoded peptide/protein that can be used for identifying specific interacting sequences (Gupta, S. et al. (2001) “MAPPING OF HIV-1 GAG EPITOPES RECOGNIZED BY POLYCLONAL ANTIBODIES USING GENE-FRAGMENT PHAGE DISPLAY SYSTEM,” Prep Biochem Biotechnol. 31(2):185-200; Kuwabara, I. et al. (1999) “MAPPING OF THE MINIMAL DOMAIN ENCODING A CONFORMATIONAL EPITOPE BY LAMBDA PHAGE SURFACE DISPLAY: FACTOR VIII INHIBITOR ANTIBODIES FROM HAEMOPHILIA A PATIENTS,” J. Immunol. Methods 224(1-2):89-99; Santi, E. et al. (2000) “BACTERIOPHAGE LAMBDA DISPLAY OF COMPLEX CDNA LIBRARIES: A NEW APPROACH TO FUNCTIONAL GENOMICS,” J. Mol. Biol. 296(2):497-508; Santini, C. et al. (1998) “EFFICIENT DISPLAY OF AN HCV CDNA EXPRESSION LIBRARY AS C-TERMINAL FUSION TO THE CAPSID PROTEIN D OF BACTERIOPHAGE LAMBDA,” J. Mol. Biol. 282(1):125-135).

Recently, a two phage system has been described for detecting or assessing protein/protein interactions using bacteriophage M13 (Pillutla, R. et al.; U.S. Patent Publn. No. 20030180718). However, the M13 system has several limitations. One limitation is that the high-density display of large protein domains on M13 is inefficient and is often associated with extensive degradation (McCafferty, J. et al. (1991) “PHAGE-ENZYMES: EXPRESSION AND AFFINITY CHROMATOGRAPHY OF FUNCTIONAL ALKALINE PHOSPHATASE ON THE SURFACE OF BACTERIOPHAGE,” Protein Eng. 4(8):955-961). Additionally, since M13 morphogenesis occurs in the periplasm, molecules that are secretion-incompetent may not be displayed by an M13 display system. For example, peptides or polypeptides that display a single or an odd number of cysteine residues (Kay, B. K. et al. (1993) “AN M13 PHAGE LIBRARY DISPLAYING RANDOM 38-AMINO-ACID PEPTIDES AS A SOURCE OF NOVEL SEQUENCES WITH AFFINITY TO SELECTED TARGETS,” Gene. 128(1):59-65) or peptides that contain a disproportionate number of charged residues, either acidic or basic (Peters, E. A. et al. (1994) “MEMBRANE INSERTION DEFECTS CAUSED BY POSITIVE CHARGES IN THE EARLY MATURE REGION OF PROTEIN PIII OF FILAMENTOUS PHAGE FD CAN BE CORRECTED BY PRLA SUPPRESSORS,” J. Bacteriol. 176(14):4296-4305) may not be displayed using such systems.

Particularly in light of the discovery that countless open reading frames (“ORFs”) of unknown function exist across many generi, efforts to discover biochemical partners of novel gene products, and to diagram and understand uncharacterized protein networks require a protein association assay that is specific, sensitive and flexible. A further need exists for such a system that would be amenable to the identification or assessment of polypeptides of substantial size. The invention described herein is directed to address these and other needs.

### SUMMARY OF THE INVENTION

A central challenge following the recent attainment of numerous genomic sequences is the realization of a comprehensive description of those protein-protein interactions responsible for governing complex communication networks and those alterations occurring in the disease states. In order to identify the components and study their interactions, the invention provides a 2-hybrid system that is based on bacteriophage λ display tools. The validity of this approach has been demonstrated by analyzing known specific interactions (i) of protein-sorting signal Ubiquitin and the CUE domain of Vsp9p, and (ii) of synthetic acidic and basic aptamers. In contrast to the available (i) Yeast 2-hybrid system, (ii) E. coli 2-hybrid system, (iii) peptide immobilization-based panning procedure, and (iv) T7 and M13 phage display systems, the approach described herein may be carried out in the absence of cellular environments (i.e., it may be conducted ex vivo). Additionally, the methods of the present invention do not require the construction of null or “knock-out” strains, membrane passage is not required, and the assay may be conducted free from harsh chemical treatments. Unlike other phage display systems, the methods of the present invention do not introduce a bias towards small domains, and can achieve a high density of display.

The methods of the present invention provide a simple way for independent verification of interactions between proteins obtained through other means, and provide an attractive complement for identifying the largest number of interactions with the lowest amount of background. The methods of the present invention also provide a flexible platform to perform library panning with single or multiple bait(s), to define proteomics, to discover inhibitors (potential drugs), and to identify and analyze macromolecular interactions that are dependent upon specific mediators.

In one aspect, the invention relates to a method of identifying or assessing a binding interaction between a target molecule and a target-binder molecule comprising the steps of: (a) forming a reaction mixture of a first lambdoid phage that displays a target molecule and a second lambdoid phage that displays a target-binder molecule under conditions permissive for a binding interaction between said target molecule and said target-binder molecule; (b) contacting said reaction mixture with host cells under conditions permissive for lambdoid phage infection of said host cells; and (c) assaying said host cells for co-infection by said first lambdoid phage and said second lambdoid phage, wherein co-infection is indicative of a binding interaction between said target molecule and said target-binder-molecule.

In another aspect, the invention relates to a method of identifying or assessing protein binding modulators comprising the steps of: (a) forming a reaction mixture comprising a first lambdoid phage that displays a target molecule and a second lambdoid phage that displays a target-binder molecule, in the presence and absence of test modulator, under conditions permissive for a binding interaction between said target molecule and said target-binder molecule; (b) contacting said reaction mixture with host cells under conditions permissive for lambdoid phage infection of said host cells; and (c) assaying said host cells for co-infection by said first lambdoid phage and said second lambdoid phage and observing the effect of the test modulator on the number of co-infections, wherein co-infection is indicative of a binding interaction between said target molecule and said target-binder-molecule, and wherein said test modulator is identified as a protein-binding modulator if the number of co-infections in the presence of the test modulator is greater or less than the number of co-infections in the absence of the test modulator.

In another aspect, the invention relates to a method of identifying or assessing a binding interaction between a target molecule and a target-binder molecule comprising the steps of: (a) mixing a first lambdoid phage preparation, said preparation comprising first lambdoid phages that display a target molecule, and a second lambdoid phage preparation, said preparation comprising second lambdoid phages that display a target-binder molecule, under conditions permissive for a binding interaction between said target molecule and said target-binder molecule; and (b) assaying for phage complex formation between at least one first lambdoid phage and at least one second lambdoid phage, wherein said phage complex formation is indicative of a binding interaction between said target molecule and said target-binder-molecule.

### BRIEF DESCRIPTION OF THE FIGURES

FIG. 1, Panel A is a schematic diagram of one embodiment of the invention. A first lambda phage library with lambda phage particles (A) that display target molecules (shown as dark gray protrusions) and a second lambda phage library with lambda phage particles (B) that display target-binder molecules (shown as light gray protrusions) are combined. When a target molecule and a target-binder molecule exhibit a protein/protein binding interaction, coinfection of a single host cell by a lambda phage particle (A) and a lambda phage particle (B) occurs. For simplicity, only one target molecule and one target-binder molecule exhibiting a binding interaction are shown. In one embodiment, each of the display phages confers resistance to an antibiotic resistance determinant such as kanamycin or chloramphenicol. When the peptides decorating the arrayed phage protein (e.g., the D protein) of the two phages undergo a protein-protein interaction, phage aggregates are formed. These phages at a very low MOI can then co-infect a cell yielding a stable multilysogen that is resistant to both antibiotics. FIG. 1, Panel B provides a diagram of the principle behind the lambdoid phage-based Two Hybrid assay for the analysis of macromolecular interactions. Rows a, b, and c illustrate the steps for potential protein recognition, potential protein association and potential dual infection of a cell resulting in a double resistant lysogen. If the two phages are not displaying a D-fusion (vector phages in rows I and II), there is no association between the two phages and only mono-resistant lysogens will result. If the phages are displaying D-fusions that do associate (Row III) or that associate through a third species (Row IV), then the phages will co-infect a cell resulting in a Kan^{r}/Cml^{r} double resistant multilysogen,

FIG. 2, Panels A, B and C provide a diagrammatic representation of different preferred vectors that may be employed in a lox-Cre recombination system for the construction of a lambda phage genome that encodes a target protein fused to the lambda gpD protein. Only relevant genes and restriction sites are shown. The maps are not to scale. The labels in the diagram are as follows: lacPO—the lac promoter-operator; RBS—a ribosome-binding site; D—segment encoding amino acid residues 1-109 of the lambda gpD protein; Stuffer—a 30 nucleotide long sequence; c-myc—a decapeptide recognized by the monoclonal antibody, 9E10; fₒᵣᵢ—the origin of replication of filamentous phage f; Amp^{r}—the β-lactamase gene (conferring resistance to ampicillin); Ori—the Co1E1 origin of replication; loxP_{wt}—a wild-type lox site; loxP₅₁₁—a lox site with mutation 511. Panel A shows the donor plasmid, pVCDcDL1, with cloning sites NheI and MluI. Panel B shows the recipient phage vector, DL1. Only some of the lambda genes are shown. Dam represents the D gene of lambda with an amber mutation. The unique XbaI site in the lambda genome used for cloning is shown. The lacZα cassette comprises lacPO, RBS and the first 58 codons of lacZ. L1 and L4 are oligonucleotide primers used for PCR-based analysis of cointegrates. Panel C shows the donor plasmid pVCDcDL3, which is similar to pVCDcDL1 but which contains, between the NheI and MluI sites, a lacZ cassette comprising lacPO, RBS and the first 148 codons of lacZ flanked by SmaI/SrfI restriction enzyme sites. Blunt-ended DNA fragments can be cloned into SmaI/SrfI-cut vector and recombinants produce white colonies on X-gal plates. T represents a universal translation stop.

FIG. 3 is a diagrammatic representation of the lox-Cre recombination process and provides a schematic of the genetic steps in the construction of embodiment of display phage for protein-protein interaction studies. The lox sites shown in black are of the recipient lambda phage vector. Cre represents the Cre recombinase. SCO represents a single crossover cointegrate. DCO represents a double crossover cointegrate. Filled arrows indicate the direction of transcription from the promoter of β-lactamase, lacZ and λD gene. L1 and L4 are oligonucleotides used for the PCR-based analysis of cointegrates. Only one of the possible recombination pathways is shown (i.e. a first crossover at loxP_{wt} followed by second crossover at loxP₅₁₁). The other pathway (i.e. a first crossover at loxP₅₁₁ followed by second crossover at loxP_{wt}) will yield the same product. D-fusions are generated in the pDC3 plasmid by standard genetic manipulations. Recombineering of the vector phages occurs within a host cell containing the Cre-Lox recombination function of the P1 phage. Cre-promoted site-specific recombination at the wild type Lox (wtLox) and mutant Lox (mutLox) sites transfers the wtD-fusion construct from the pDC3 vector into the phage genome. Phages resulting from this process are selected for Ampr and wtD production.

FIG. 4 shows that pre-association with a binding partner significantly increases the number of stable monoresistant multi-lysogens. Mixing phages with a binding partner prior to cell infection increases the number of monoresistant lysogens at lower MOI's as a result of aggregation. Specific aliquots of vector or display phage lysates were incubated at room temperature for 5 minutes to allow for protein-protein interactions, followed by dilution into salted adsorption buffer. After 15 min, freshly cultured 1×10⁸ E. coli LE392 cells were added to the phage mixture. The reactions are plated on either chloramphenicol or kanamycin agar plates and incubated over night at 32° C. With only 1 antibiotic being selected, the total lysogen count includes those cells infected with 2 of the same phage, one of each of the phages and a single phage. Panel A illustrates the formation of monoresistant multi-lysogens. Panel B represents the monoresistant lysogen count from the vector phages (A2 and A3). Note that the largest Y-axis value is much smaller than that of the Y-axis values in the following panels. Panel C the λD-Acid and λD-Base phages. Panel D shows complex formation by the λD-CUE and λD-Ubiquitin phages. During the incubation period, the formation of aggregates of λ D-Base on λ D-Acid results cells being infected with >1 phage. The result is a high number of Kan^{r} and Cml^{r} stable lysogens that contain 2+ phage gemones at a low MOI. Panel E shows the contribution of hetero-co-infection by selection for Cml^{r}/Kan^{r} cells. The number of colonies counted reveals that co-infection by binding partners begins to contribute to stable lysogen formation at an MOI far below 1 but does not begin for vector phages λ-A2:λ-A3 (diamonds) until the number of phage exceeds the number of cells (an MOI>1). In contrast, the formation of double resistant multilysogens for the interacting pairs of Acid/Base (squares) begins at an MOI of ˜0.0003 and for λD-CUE:λD-Ubiquitin (triangles) at an MOI of ˜0.03. The λD-Acid:λD-Base pair appears to have a higher efficiency of infection.

FIG. 5 shows that display phage association differs at increasing MOI's based upon the strength of binding between their expressed peptides. Specific aliquots of vector or display phage lysates were incubated at room temperature for 5 minutes to allow for phage-phage interactions, followed by dilution into salted adsorption buffer. After 15 min, freshly cultured E. coli LE392 cells (to the final MOI designated) were added to the phage mixture. The infected cells were allotted 45 min at room temperature to express the antibiotic resistance markers. The reactions were then spread on selective plates and incubated over night at 32° C. The data is presented as the number of double resistant colonies counted. The stronger Acid/Base (diamonds) and CUE/Ubiquitin (squares) pairs produced increasing numbers of multilysogens, with a maximum saturation point reached of 25% of the available cells. The less specific Ubiquitin/Ubiquitin (asterisks) and CUE/Acid (triangles) pairs resulted in fewer multilysogens and a lower point of maximum infectivity. The vector pair A2/A3 (X's) showed little to no interaction, with only accidental double-infection occurring only at high MOI's where the number of input phage far outnumber the available host cells.

FIG. 6 shows the titration of Acid/Base Association by the Acidic and Basic Aptamers. Lambda display phages are used to co-infect cells at an MOI of 0.0025. In the presence of increasing concentrations of the Acidic (triangles) or Basic (square) Aptamer (0.001 μM to 5 μM), titration of Acid/Base association is scored by the loss of double resistant multilysogen formation. The point [0,0] representing the maximum number of lysogens for this given MOI. Here, the maximum number of double resistant multilysogens is approximately 150 per 8×10⁵ input phages. Inhibition by both aptamers is approximately equal, with an IC₅₀ of ˜0.01 μM.

FIG. 7 shows titration CUE/Ubiquitin by free wtUbiquitin and wtCUE, but not mutant CUE. Lambda display phages are used to co-infect cells at an MOI of 0.04. In the presence of increasing concentrations of the wtUbiquitin (diamonds), wt CUE (squares) or mutant CUEM419D (triangles) protein is added to the phage pair, and positive λD-CUE:λD-Ubiquitin association is scored by the loss of double resistant multilysogen formation. The results are tabulated as the number of colonies counted, with the point [0,0] representing the maximum number of lysogens for this given MOI. Here, the maximum number of double resistant multilysogens is approximately 120 per 8×10⁵ input phages. Inhibition by wt Ubiquitin is approximately 0.003 μM, whereas the IC50 for CUE is ˜0.0008. There is no titration point for the non-binding mutant CUEM419D.

### DESCRIPTION OF THE PREFERRED EMBODIMENTS

The present invention relates to methods and compositions for the identification and/or assessment of protein/protein interactions, and in particular to methods and compositions for accomplishing the high-throughput detection of interactions of proteins displayed on the surfaces of lambdoid bacteriophage particles. The objective of any display system is to identify the highest number of binding partners with lowest possible background. Phage display is quickly becoming a tool of choice to study in functional genomic studies as it continually proves to be a viable alternative to the yeast 2-Hybrid system (Auerbach, D. et al. (2002) “THE POST-GENOMIC ERA OF INTERACTIVE PROTEOMICS: FACTS AND PERSPECTIVES,” Proteomics 2(6):611-23) and bacterial 2-Hybrid system (Simon, L. et al. (2004) “PROTEIN-PROTEIN INTERACTIONS METHODS AND APPLICATIONS,” Methods in Molecular Biology 261: 231-246; Kirsch, M. et al. (2005) “PARAMETERS AFFECTING THE DISPLAY OF ANTIBODIES ON PHAGE,” J. Immunol. Methods. 301(1-2):173-85) for studying protein association. However, this technique has previously been limited to bio-panning (Kay, B. K. et al. (1996) “Phage Display Of Peptides And Proteins. Academic Press, NY) against immobilized targets. Lambda display has historically been used only against immobilized prey, wherein the bound phage are eluted and characterized, and the next logical step in the evolution of lambda display is the development of a 2-Hybrid strategy based upon display from its abundant D head protein.

The present invention provides a lambdoid bacteriophage-based version of the 2-Hybrid system that is well suited to compliment and improve upon existing tools for studying protein association. The present invention provides a 2-Hybrid system that is inherently low in background and false positives thereby biasing the results towards real interactions, or ‘true positives’. This is in stark contrast to the Yeast 2-Hybrid system that has been faulted with presenting almost 50% false positive rate due to aberrant activation of gene transcription in the absence of bait and prey interaction (Figeys, D. (2004) “COMBINING DIFFERENT ‘OMICS’ TECHNOLOGIES TO MAP AND VALIDATE PROTEIN-PROTEIN INTERACTIONS IN HUMANS,” Brief Funct. Genomic Proteomic. 2(4):357-365; Kofler, M. et al. (2005) “NOVEL INTERACTION PARTNERS OF THE CD2BP2-GYF DOMAIN,” J. Biol. Chem. 280(39):33397-402). The interrogation provided by the present invention, unlike the Yeast 2-Hybrid system, may be conducted ex vivo. Thus, there is no need to create null or knock-out strains, interactions occur free from interference of cellular contents, every aspect of the reaction conditions can be controlled to a high degree, and one can assay the specific effect(s) of particular peptide(s) or other species on potential interactions. The present invention thus holds enormous value for assaying suspected binding partners, presenting recalcitrant proteins (i.e. scFv), performing interrogations of association and inhibition kinetics, providing an economical drug discovery platform and performing library screening and antigen optimization procedures (Chowdhury, P. S. et al. (1999) “IMPROVING ANTIBODY AFFINITY BY MIMICKING SOMATIC HYPERMUTATION IN VITRO,” Nat. Biotechnol. 17(6):568-572) in which selection is naturally driven by the strength, affinity and/or stability of the protein associations.

As used herein, a “lambdoid” phage is a bacteriophage (λ) lambda or a derivative or variant thereof. Lambdoid phages that may be employed include, for example, phage lambda (λ), and variants and derivatives of phage λ (especially lambdoid phages having non-λ immunity. Examples of variants and derivatives of phage λ include phage 21, phage 82, phage φ80, phage φ81, phage Hong Kong, phage 424 and phage 434. In a preferred embodiment of the invention, the type of lambdoid bacteriophage employed is the bacteriophage lambda. The bacteriophage lambda comprises an icosahedral head or capsid with a radius of 30 nm and a flexible tail 150 nm long ending in a tapered basal part and a single tail fiber. The genome of lambdoid bacteriophages comprise linear DNA with cohesive ends, so as to facilitate the circularization of the phage DNA. The DNA is found in the capsid head, and the right end of the DNA, as defined by the genetic map, protrudes into the upper third of the phage's tail structure. The use of lambdoid phages in a display assay allows great flexibility and broad application, and is bolstered by a multitude of successes in biopanning (Ansuini, H. et al. (2002) “BIOTIN-TAGGED CDNA EXPRESSION LIBRARIES DISPLAYED ON LAMBDA PHAGE: A NEW TOOL FOR THE SELECTION OF NATURAL PROTEIN LIGANDS,” Nucleic Acids Res. 30(15):e78; Cicchini, C. et al. (2002) “SEARCHING FOR DNA-PROTEIN INTERACTIONS BY LAMBDA PHAGE DISPLAY,” J. Mol. Biol. 322(4):697-706).

The preferred methods of the present invention involve infecting host cells with such lambdoid phages at low, and preferably extremely low, multiplicities of infection (“MOI”). Thus, the MOI is preferably less than 1, more preferably less than 0.5, still more preferably less than 0.1, still more preferably less than 0.05, still more preferably less than 0.01. Unique to lambdoid phages, infection of a cell culture by lambda at an MOI below 1 will not sustain lysogenic infection (Oppenheim, A. B. et al. (2005) “SWITCHES IN BACTERIOPHAGE LAMBDA DEVELOPMENT,” Annu Rev Genet. (Epub ahead of print); Svenningsen, S. L. (2005) “ON THE ROLE OF CRO IN LAMBDA PROPHAGE INDUCTION,” Proc. Natl. Acad. Sci. USA 102(12):4465-4469; Kobiler, O. et al. (2005) “QUANTITATIVE KINETIC ANALYSIS OF THE BACTERIOPHAGE LAMBDA GENETIC NETWORK,” Proc. Natl. Acad Sci USA. 102(12):4470-4475), and further only at MOI's>2 will two lambdoid phage infect the same cell; and here the dual-infection is merely coincidental. When two or more phages are associated at their heads through displayed protein interactions they are forced to co-infect a cell and this maintains the lysogenic state even at extremely low MOI's (10⁻² to 10⁻⁶). The presence of two or more lambda genomes stabilizes the lysogenic state because two genomes produce together high levels of cI, causing lysogeny to be the preferred state even in light of high levels of protease present in the log phase cells used. Phages displaying potential binding partners are assayed at an MOI far below 1, therefore only those fusion display phages involved in a protein-protein association can simultaneously infect a cell. Interactions with affinities as low as 50 μM are sufficient to be detected (Zucconi, A. et al. (2001) “SELECTION OF LIGANDS BY PANNING OF DOMAIN LIBRARIES DISPLAYED ON PHAGE LAMBDA REVEALS NEW POTENTIAL PARTNERS OF SYNAPTOJANIN,” J. Mol. Biol. 2001 307(5):1329-1339.

Thus, in a preferred embodiment of the methods of the present invention, the genomes of lambdoid phages are engineered to express a fusion protein composed of an arrayed phage protein and a target protein. The non-essential major capsid protein D has been an attractive target for the expression of foreign proteins on λ because it has many advantages over other phage proteins used for display. Protein D functions to stabilize the head following binding of the gpE subunits during expansion of the head, and is added to the lattice in the last stage of head maturation. Therefore, unlike with M13, degradation of fusion display proteins (Terry, T. D. et al. (1997) “ACCESSIBILITY OF PEPTIDES DISPLAYED ON FILAMENTOUS BACTERIOPHAGE VIRIONS: SUSCEPTIBILITY TO PROTEINASES,” Biol. Chem. 378(6):523-530) and the structure or nature of a display protein (hydrophobicity, charge factor, disulfide bonds or transmembrane domain) are not of concern because the λ D protein is assembled onto the phage following head formation. M13 also has limits on the display of large domains (Malik, P. et al. (1996) “ROLE OF CAPSID STRUCTURE AND MEMBRANE PROTEIN PROCESSING IN DETERMINING THE SIZE AND COPY NUMBER OF PEPTIDES DISPLAYED ON THE MAJOR COAT PROTEIN OF FILAMENTOUS BACTERIOPHAGE,” J. Mol. Biol. 260(1):9-21) and cytoplasmic species because the fusion protein must be able to be passed through a membrane as it is moved from the cytoplasm to the periplasm. Protein size is less of an issue that with T7 since large proteins (1000+ amino acids long) have been successfully displayed as functional D-fusions; therefore one can carry out assays under conditions that do not have bias towards smaller domains (Zucconi, A. et al. (2001) “SELECTION OF LIGANDS BY PANNING OF DOMAIN LIBRARIES DISPLAYED ON PHAGE LAMBDA REVEALS NEW POTENTIAL PARTNERS OF SYNAPTOJANIN,” J. Mol. Biol. 2001 307(5):1329-1339). Also unique to the λ D protein is the high number of copies of present per virion head (>400) each able to serve as a scaffold for presentation, awarding a tremendous potential for interactions between display fusion proteins. High multivalency is crucial when using bait or prey of unknown affinity or concentration (such as antibodies from patient sera) (Folgori, A. et al. (1994) “A GENERAL STRATEGY TO IDENTIFY MIMOTOPES OF PATHOLOGICAL ANTIGENS USING ONLY RANDOM PEPTIDE LIBRARIES AND HUMAN SERA,” EMBO J. 13(9):2236-43). Additionally, with the high resolution 3D structure of the D protein solved (Yang, F. et al. (2000) “NOVEL FOLD AND CAPSID-BINDING PROPERTIES OF THE LAMBDA-PHAGE DISPLAY PLATFORM PROTEIN GPD,” Nat Struct Biol Mar; 7(3):230-7), rational design can be applied to optimize fusion of a target protein to gain a maximal degree of freedom for association.

In a preferred embodiment of the invention, a first and second lambdoid phage preparation is provided. The first lambdoid phage preparation comprises lambdoid phages that display a “target” molecule, or a population of the same or different “target” molecules, on the surface of the phage particles. The second lambdoid phage preparation comprises lambdoid phages that display a “target-binder” molecule, or a population of the same or different “target-binder” molecules, on the surface of the phage particles. A phage particle may have only a single target or target binder molecule on its surface, but more preferably, will have more than a single target or target binder molecule on its surface. Likewise, a phage particle may have only a single molecular species of target or target binder molecule on its surface or may have multiple molecular species of target or target binder molecule on its surface. Either or both of the lambdoid phage preparations may comprise a library of phages that array different target molecules. The size of any such library may be small (having fewer than 1,000 members), moderate in size (having 10,000-100,000 members) or larger (10⁵, 10⁶, 10⁷, 10⁸ members or more) in size. The lambdoid phages of the first and second lambdoid phage preparations are incubated together under conditions that are permissive for a binding interaction to occur between the target molecule(s) and the target-binder molecule(s) so as to form a phage complex comprising a lambdoid phage of the first lambdoid phage preparation and a lambdoid phage of the second lambdoid phage preparation. The mixture is then assayed for phage complex formation, wherein phage complex formation is indicative of a binding interaction between the target molecule and the target-binder molecule. Additional phage preparations (e.g., a third lambdoid phage preparation, a fourth lambdoid phage preparation, etc.) may be employed in order to detect binding interactions involving three proteins, binding interactions involving four proteins, or higher order protein interactions.

In one embodiment of the invention, phage complex formation is assayed via the detection of co-infection of a host cell by phages of the first and second phage preparations. For example, the lambdoid phages of the first lambdoid phage preparation and the second lambdoid phage preparation may contain genetic markers that allow for the identification of cells that have been co-infected by phages of the first and second phage preparations. In an alternate embodiment of the invention, phage complex formation may be assayed via the physical detection of phage complexes. In one such embodiment, the binding of the first and second lambdoid phages will be detected by the recovery of cells that are lysogenic for both phages. In such an embodiment, it is desirable to employ phages having the same immunity (e.g., both the first and second lambdoid phages being of lambda immunity, etc.) In an alternative embodiment of the invention, the binding of the first and second lambdoid phages will be detected by the production of lytic phage. In such an embodiment, the employed first and second phages may have either the same or different immunity (e.g., the first lambdoid phage may be immunity lambda, and the second lambdoid phage may be of immunity lambda or immunity 434, etc.).

A second embodiment of the invention relates to the recognition of a novel and efficient method for creating a lambdoid phage that displays a target molecule or a target-binder molecule, or libraries of such molecules, via homologous recombination between a starter phage and one or more double-stranded or single-stranded donor nucleic acid molecules that encode the target or target-binder molecules so as to incorporate the target molecule and/or target-binder molecules as part of a fusion protein with one of the proteins displayed by the lambdoid phage particle.

The λ-based two hybrid system of the present invention possesses intrinsic advantages over other systems. In some embodiments, however, the expression of a lambdoid receptor from the D protein may not be fully compatible with the dual infection property. Additional modifications may be required in order to use the invention to detect a protease capable of digesting the lambdoid head or tail proteins. Three other potential drawbacks that can be corrected for are cloning of very large (>3 Kb) genes, expression of eukaryotic proteins that require specific modifications for proper activity (e.g., phosphorylation, farnesylation, etc.) or the attempted expression of an anti-bacterial agent from the phage head. The former can be accommodated by removal of 2,000 base pairs of non-essential genetic material from the phage genome. However, larger insertions (e.g. 2,000-5,000 base pairs, 5,000-10,000, or more than 10,000 base pairs) can be accommodated by deleting even more non-essential genetic material from the phage genome. Both the cloning and modification issues can be addressed by producing the D-fusion protein in a separate cell (bacterial or eukaryotic) and providing the D-fusion to lambda in trans (Zanghi, C. N. (2005) “A SIMPLE METHOD FOR DISPLAYING RECALCITRANT PROTEINS ON THE SURFACE OF BACTERIOPHAGE LAMBDA,” Nucleic Acids Research 33(18):160). The latter can be overcome by co-expression of an antibiotic resistance marker in the host cells, since the presence of a plasmid will not interfere with transduction or expression from the infecting phages genomes. Finally, the expression and study of proteins whose activity is contained within their amino terminal end can be cloned into a version of pDC3 that allows for fusion of genes of interest to gene d at their 3′ end.

In a preferred embodiment, the target molecule and the target-binder molecule comprise peptides or polypeptides that are displayed on the outer surface of the lambdoid phage particle via the incorporation of the target molecule or the target-binder molecule into a display protein. As used herein, a “display protein” refers to any lambdoid phage protein that is accessible on the exterior surface of the lambdoid phage particle. As used herein, a target molecule or a target-binder molecule is “incorporated” into a display protein when the target molecule or the target-binder molecule is expressed as a fusion protein with at least a portion of a display protein to form a display fusion protein. In addition to the target molecule or the target-binder molecule and the display protein, the display fusion protein may also comprise auxiliary amino acid sequences. Auxiliary amino acid sequences may be, for example, sequences that are inserted between the target molecule or the target-binder molecule and the display protein to enhance the display characteristics of the target molecule or the target-binder molecule. Auxiliary amino acid sequences may also be, for example, tag sequences that facilitate isolation of the display fusion protein or the target molecule or the target-binder molecules. The target molecule or the target-binder molecule may be incorporated at either the amino-terminus, the carboxy-terminus, or at an interior portion of the display fusion protein.

Bacteriophage lambda is a preferred lambdoid phage. A preferred lambda display protein is the λgpD protein, an 11.4 kDa capsid stabilizing protein. During morphogenesis, lambda DNA is packaged in the prohead shell that expands and undergoes an irreversible conformational change that allows the λgpD protein to bind to the prohead (Wurtz, M. et al. (1976) “SURFACE STRUCTURE OF IN VITRO ASSEMBLED BACTERIOPHAGE LAMBDA POLYHEADS,” J. Mol. Biol. 101(1):39-56; Imber, R. et al. (1980) “OUTER SURFACE PROTEIN OF BACTERIOPHAGE LAMBDA,” J. Mol. Biol. 139(3):277-295). Cryoelectron microscopy has shown that gpD is exposed on the surface of the capsid (Dokland, T. et al. (1993) “STRUCTURAL TRANSITIONS DURING MATURATION OF BACTERIOPHAGE LAMBDA CAPSIDS,” J. Mol. Biol. 233(4):682-694). Typically, a capsid contains approximately 400 copies of the gpD protein. Trimers of gpD bind to underlying molecules of gpE that form the capsid shell. The first 15 amino acids of gpD must contact gpE since deletion derivatives that remove these amino acids can still fold correctly but will not bind lambda D-heads (Yang, F. et al. (2000) “NOVEL FOLD AND CAPSID-BINDING PROPERTIES OF THE-PHAGE DISPLAY PLATFORM PROTEIN GPD,” Nat. Struct. Biol. 7(3):230-237). Although the lambda crystal structure shows that both the amino and carboxy termini of gpD appear to point downward towards the capsid interior, peptides and proteins fused to gpD are nevertheless accessible at the surface, possibly facilitated by linkers that join gpD and the fusion partner. Polypeptides fused at either the amino- or carboxy terminus of gpD are displayed (Sternberg, N. et al. (1995) “DISPLAY OF PEPTIDES AND PROTEINS ON THE SURFACE OF BACTERIOPHAGE LAMBDA,” Proc. Natl. Acad. Sci. USA 92(5):1609-1613; Mikawa, Y. G. et al. (1996) “SURFACE DISPLAY OF PROTEINS ON BACTERIOPHAGE LAMBDA HEADS,” J. Mol. Biol. 262(1):21-30).

A second preferred lambda display protein is the lambda tail protein gpV, the product of the λV gene. The lambda tail consists mainly of a tube of 32 disks each composed of six gpV protein units. Genetic and biochemical analyses indicate that the carboxy terminal portion of the protein is dispensable (Katsura, I. (1976) “ISOLATION OF LAMBDA PROPHAGE MUTANTS DEFECTIVE IN STRUCTURAL GENES: THEIR USE FOR THE STUDY OF BACTERIOPHAGE MORPHOGENESIS,” Mol. Gen. Genet. 148(1):31-42). Electron micrographs of the hexamer rings formed by gpV show that the carboxy terminal deletion mutants lack protrusions on the outer surface when compared with wild-type gpV preparations (Katsura, I. (1981) “STRUCTURE AND FUNCTION OF THE MAJOR TAIL PROTEIN OF BACTERIOPHAGE λ MUTANTS HAVING SMALL MAJOR TAIL PROTEIN MOLECULES IN THEIR VIRION,” J. Mol. Biol. 146(4):493-512). Despite the gpV carboxy deletions, such phages are viable. A variety of accessibly displayed carboxy terminal fusions to gpV have been described (Maruyama, I. N. et al. (1994) “LAMBDA FOO: A LAMBDA PHAGE VECTOR FOR THE EXPRESSION OF FOREIGN PROTEINS,” Proc. Natl. Acad. Sci. USA 91(17):8273-8277; Dunn, I. S. (1995) “ASSEMBLY OF FUNCTIONAL BACTERIOPHAGE LAMBDA VIRIONS INCORPORATING C-TERMINAL PEPTIDE OR PROTEIN FUSIONS WITH THE MAJOR TAIL PROTEIN,” J. Mol. Biol. 248(3):497-506; Dunn, I. S. (1996) “IN VITRO ALPHA-COMPLEMENTATION OF BETA-GALACTOSIDASE ON A BACTERIOPHAGE SURFACE,” Eur. J. Biochem. 242(3):720-726).

In one embodiment of the invention, at least one of or both of the target molecule and the target-binder molecule are incorporated at the amino- or carboxy-terminus of the lambda gpD protein or at the carboxy-terminus of the lambda gpV protein. In a preferred embodiment, at least one of or both of the target molecule and the target-binder molecule are incorporated at the carboxy-terminus of the lambda gpD protein. As used herein, incorporated at the amino-terminus or the carboxy-terminus refers to the general location of the target molecule or the target-binder molecule as part of the display fusion protein and does not indicate a requirement that the target molecule or the target-binder molecule comprise the actual amino-terminus or the carboxy-terminus of the display fusion protein.

In one embodiment of the invention, greater than about 10%, preferably greater than about 25%, more preferably greater than about 50% and most preferably greater than about 90% phage particles of the first lambdoid phage preparation and/or the second lambdoid phage preparation will have an average number of target molecules or target-binder molecules per phage particle of greater than about 50, preferably greater than about 100, more preferably greater than about 175, and most preferably greater than about 400 target molecules or target-binder molecules per phage particle.

Preferably, the first lambdoid phage preparation and/or the second lambdoid phage preparation will comprise phage particles having target/target-binder molecules possessing an average (mean) length of greater than about 50 amino acids, preferably greater than about 75 amino acids, more preferably greater than about 100 amino acids, and most preferably greater than about 150 amino acids.

Thus, in one preferred embodiment, greater than 90% phage particles of either or both the first lambdoid phage preparation and/or the second lambdoid phage preparation possess greater than 350 target molecules or target-binder molecules per phage particle, wherein the average amino acid length of the target molecules or target binder molecules is greater than 50 amino acids. Such target molecules (or target binders) may be identical or different from one another.

The first lambdoid phage and the second lambdoid phage will preferably contain “genetic markers” (i.e., expressible traits) that allow for the identification of cells that have been co-infected by the first lambdoid phage and the second lambdoid phage. Preferably, the genetic markers are “selectable markers” (i.e., traits that confer a survival or propagation advantage). Co-infected cells may be identified both when the phage are in the lytic mode or in the lysogenic mode. For example, genetic markers for the identification of co-infected cells include selectable markers such as, for example, drug resistance or auxotrophic markers, or screenable markers such as, for example, fluorescence markers, etc. Genetic markers may be constitutive or they may be inducible or repressible under certain conditions. For example, genetic markers may be temperature sensitive or suppressible (e.g., amber suppressible, ochre suppressible). For certain applications, E. coli host cells may be employed that facilitate the selection of co-infected cells wherein the phage are in the lysogenic mode such as, for example, E. coli cells having hfl mutations.

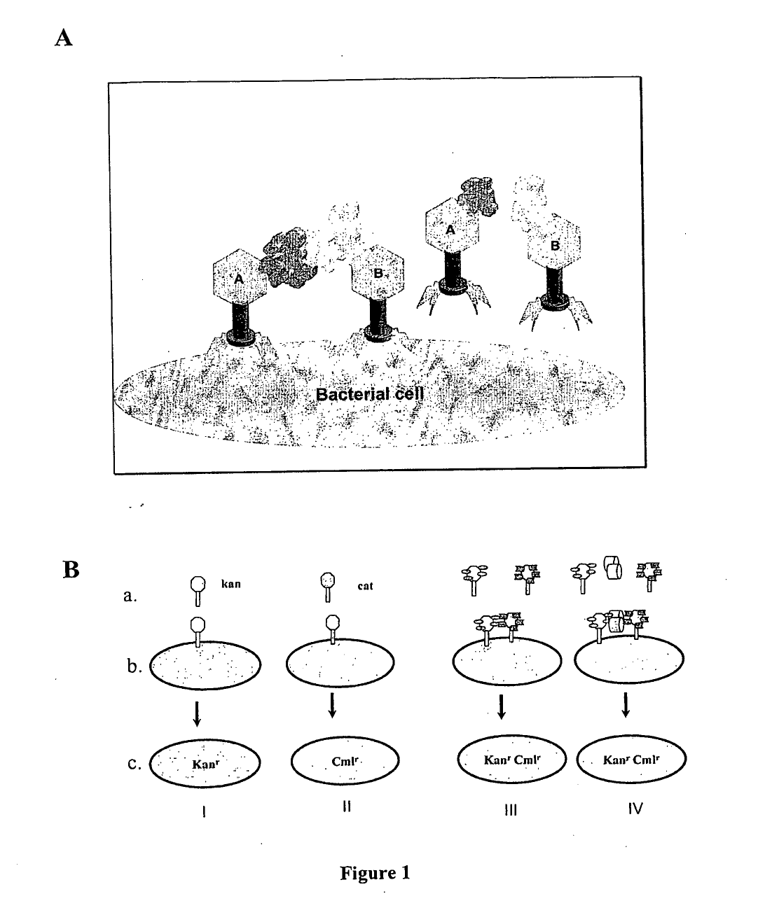

In a preferred embodiment, the first lambdoid phage possess a first genetic mutation and the second lambdoid phage possess a second genetic mutation, wherein the first and second genetic mutations render the respective phages incompetent for plaque formation with a selected host E. coli strain, and wherein the first and second genetic mutations complement each other so that plaque formation may result from the co-infection of such host strain by the first lambdoid phage and the second lambdoid phage. Thus, binding pairs and co-infection of a single cell by a phage complex may be identified by the formation of a bacteriophage plaque (FIG. 1, Panel A, FIG. 1, Panel B). Plaque formation may be assayed, for example, using the plate method of Davis et al. (in Advanced Bacterial Genetics (1980) Cold Spring Harbor Laboratory, Cold Spring Harbor, N.Y., p. 71).

Preferably, each first lambdoid phage and each second lambdoid phage will possess genetic markers that facilitate the identification of the two phages from co-infected cells, plaques, or lysates. These genetic markers may be the same or different than the markers for the identification of co-infected cells.

### The Target and Target-Binder Molecules

The target and target-binder molecules of the invention of the invention may be any peptide, polypeptide, or protein. Such molecules may, for example, be receptors, receptor ligands, other ligand, enzymes, chemokines, antibody fragments, etc.

Bacteriophage displaying the target molecule or the target-binder molecule may be present in a purified phage preparation (i.e., a phage preparation in which all phage of the preparation display the same target molecule or the same target-binder molecule), in a related phage preparation (i.e., a phage preparation in which all phage of the preparation display variants or derivatives of a particular target molecule or target-binder molecule), in an unrelated phage preparation (i.e., a phage preparation in which some of the phage of the preparation display target molecules or target-binder molecules that are not the same as the target molecules or target-binder molecules displayed by other phage of the preparation, or are not variants or derivatives of such phage); the phage preparations of the present invention may comprise mixtures or combinations of purified, related and/or unrelated phage preparations. As indicated above, in some embodiments, the phage preparation may comprise a library whose individual members display any one or more of a variety of different target molecules or target-binder molecules.

In one embodiment of the invention, either or both of the target molecule and the target-binder molecule may comprise expression products of a genomic or cDNA library. In such an embodiment, such a DNA molecule is inserted into the lambda genome so that the protein encoded by the molecule is expressed as a display fusion protein. The DNA molecule that is inserted into the lambda genome may be a full-length DNA molecule (i.e., encoding a full-length protein) may be less than full-length, or may encode additional amino acid residues, peptide domains, etc. The DNA libraries may be constructed using techniques well know the art or they may be purchased from a variety of commercial sources. The DNA libraries employed may be subtractive cDNA libraries (Schraml, P. et al. (1993) “cDNA SUBTRACTION LIBRARY CONSTRUCTION USING A MAGNET-ASSISTED SUBTRACTION TECHNIQUE (MAST),” Trends Genet. 9(3):70-71) and/or normalized cDNA libraries (Bonaldo, M. F. et al. (1996) “NORMALIZATION AND SUBTRACTION: TWO APPROACHES TO FACILITATE GENE DISCOVERY,” Genome Res. 6(9):791-806). Of the numerous methods for constructing cDNA libraries, the approach based on methods described in Gubler, U. et al. (1983) (“A SIMPLE AND VERY EFFICIENT METHOD FOR GENERATING CDNA LIBRARIES,” Gene 25(2-3):263-269) is the most widely used. Alternatively, commercially available cDNA libraries may be obtained from, for example, Clontech (Palo Alto, Calif.).

In another embodiment of the invention, either or both of the target molecule and the target-binder molecule may comprise a member of a library of random peptides, and the first or second lambdoid preparations may array a library of different target/target binder molecules. In such a case, DNA molecules encoding random peptides may be inserted into the lambda genome so that the proteins that they encode can be expressed as a display fusion protein. Random peptide libraries can be designed according to methods generally known to those of skill in the art (see, e.g., Dower et al.; U.S. Pat. No. 5,723,286). DNA libraries that encode random peptides may alternatively be obtained commercially from, for example, New England Biolabs (Beverly, Mass.).

In another embodiment of the invention, either or both of the target molecule and the target-binder molecule may comprise a member of libraries of random or selective mutations of a particular peptide or polypeptide or of a particular set of peptides or polypeptides. Random mutations of a particular molecule may be generated, for example, using the following techniques: DNA shuffling as described by Stemmer, W. P. (1994) (“RAPID EVOLUTION OF A PROTEIN IN VITRO BY DNA SHUFFLING,” Nature 370(6488):389-391); error prone amplification as described by Bartel, D. P. et al. (1993) (“ISOLATION OF NEW RIBOZYMES FROM A LARGE POOL OF RANDOM SEQUENCES,” Science. 261(5127):1411-1418); cassette mutagenesis as described by Hutchison et al. (1991), Methods in Enzymology 202:356-390). Selective mutations at predetermined sites may be performed using standard molecular biological techniques (Sambrook et al., Molecular Cloning: A Laboratory Manual, Cold Spring Harbor Press, 1989).

In another embodiment of the invention, the target molecule or the target-binder molecule may comprise an antibody library, preferably a single chain FV (scFV) antibody library.

### The Process

In one embodiment, the invention involves the mixing of a first lambdoid phage of a first lambdoid phage preparation and a second lambdoid phage of a second lambdoid phage preparation in a pre-incubation step to allow for the binding of target molecules with target-binder molecules to form a phage complex. The pre-incubation mixture is then contacted with the host cells. The pre-incubation time and the pre-incubation conditions may be optimized for a particular binding pair of interest according to principles well known in the art. Preferably, the temperature range for the pre-incubation will range from room temperature or 28° C. to 42° C., more preferably 30° C. to 37° C., and most preferably about 30° C. The pre-incubation time will preferably range between a few minutes and several hours. The preferred pH value would be neutral. It is also contemplated as an aspect of the invention that the first lambdoid phage, the second lambdoid phage, and the host cells may be mixed together simultaneously, and the conditions for simultaneous mixing and incubation are preferably similar to those described above for the pre-incubation period.

The first lambdoid phage and the second lambdoid phage, including any resulting phage complexes, are mixed with a large excess of host cells. Host cells that are co-infected by the first lambdoid phage and the second lambdoid phage are selected. Preferably, a low multiplicity of infection (i.e., 1 or less, and preferably 0.1 or less, and more preferably 0.01 or less, and most preferably 0.001 or less) is employed, so that the large excess of host cells (versus the number of total phage) will minimize the possibility of random co-infection of a host cell by both a first lambdoid phage and a second lambdoid phage. In a preferred embodiment, cells are plated at a density of about 1−2×10⁸ cells/ml on a solid surface (i.e. agar) and co-infected cells are identified, preferably by the formation of plaques at the locus of co-infected cells. Once co-infected cells are identified, the first lambdoid phage and the second lambdoid phage that formed the phage complex may be recovered and purified using techniques known in the art. The associated target molecules and target-binder molecules may also be identified using techniques well known in the art.

In a further embodiment of the invention, a first phage and a second phage are mixed under conditions as described above, and any formed phage complex is identified via a non-co-infection assay. A “non-co-infection assay,” as used herein, refers to an assay that identifies phage complex formation via a method other than the identification of a host cell co-infection event. For example, phage complex formation may be assayed via the isolation of phage complexes via centrifugation, size exclusion chromatography, dialysis, affinity chromatography, or filtration. In one embodiment, phage complex formation may be observed or identified via the use of marker tags that are physically linked to the phage particles. In one embodiment, the marker tags may be detectable marker tags such as, for example, organic dyes, fluorophores, fluorescent proteins, quantum dots, preferably semi-conductor quantum dots, or radioactive isotopes. The use of quantum dots is described, for example, in Gao, X. et al. (2003) (“MOLECULAR PROFILING OF SINGLE CELLS AND TISSUE SPECIMENS WITH QUANTUM DOTS,” Trends Biotechnol. 21(9):371-373). In one example, the first phage and the second phage will be labeled with different colored quantum dots and phage complex formation will be monitored via spectroscopic methods. In a further embodiment, marker tags may be ligand marker tags, wherein a “ligand marker tag” as used herein is defined as a marker tag comprising one member of a specific binding pair. Thus, in another example, one of the first or second phage will contain a ligand marker and the other phage will contain a marker tag such as a quantum dot. Phage complexes may be physically isolated from a liquid phase using a solid phase that comprises a binding agent for the ligand marker tag. Phage complexes may be identified via detection of the quantum dot on the solid phase.

In one embodiment of the invention, the first lambdoid phage is immobilized on a solid support and the solid support is incubated with a liquid phase containing the second lambdoid phage under conditions permissible for a binding interaction between the target molecule of the first phage and the target-binder molecule of the second phage. The detection of phage complex formation (i.e. the detection of a binding interaction between the target molecule and the target-binder molecule) is then accomplished via the detection of solid-phase bound second lambdoid phage. Detection of solid-phase bound second lambdoid phage may be accomplished, for example, via the use of marker tags that are attached to the second lambdoid phage. For example, the marker tag may be a detectable marker tag or a ligand marker tag, wherein the ligand marker tag binds directly or indirectly to a detectable marker. In one specific example, the first lambdoid phage is immobilized to a solid support via a technique known in the art as “plaque lifts.”

A distinct advantage of the methods of the invention over the prior art is that because the protein/protein binding interactions occur ex vivo, binding conditions for binding of the target molecule with the target-binder molecule may be varied to select for populations of binding pairs having different binding affinity. Binding conditions that may be varied include the ratio of target molecules to target-binder molecules; incubation time for the binding pairs; temperature, pH, ionic strength of the binding solution; inclusion or exclusion of competing binding agents, etc.

In one example, increasing the number of phage expressing target-binder molecules with respect to the number of phage expressing target molecules enhances the recovery of binding pairs with higher affinity. In another example, increasing the incubation time of phage expressing target molecules with phage expressing target-binder molecules enhances the recovery of binding pairs with higher affinity. In another example, increasing the stringency of the incubation condition by increasing the temperature, ionic strength, divalent cation concentration or volume of the incubation mixture enhances the recovery of binding pairs with higher affinity. Variations in pH will also affect the selection of high affinity binding pairs versus the selection of low affinity binding pairs. In another example, the inclusion of competing binding agent for the target molecules will enhance the recovery of target molecule/target-binder molecule pairs with higher binding affinity.

### Incorporation of the Target Molecules

Target molecules and target-binder molecules may be incorporated into the lambdoid phage display proteins to form display fusion proteins using a variety of recombinant DNA techniques known in the art. For example, target molecules and target-binder molecules may be incorporated into the display proteins using direct cloning techniques well known in the art to incorporate the DNA encoding the target molecule or the target-binder molecule into the genome of the lambdoid phage. Alternatively, the target molecules or target-binder molecules may be incorporated into the genome of the lambdoid phage by in vivo recombination. For example, one preferred method is the in vivo high-efficiency lox-Cre recombination system described in Example 1 herein, and also described in Gupta et al. (2003)(“HIGH-DENSITY FUNCTIONAL DISPLAY OF PROTEINS ON BACTERIOPHAGE LAMBDA,” J. Mol. Biol. 334(2):241-254) and in WO 03/096969.

The Cre-Lox recombinanse system has been extensively studied and characterized (see, Sternberg, N. et al. (1981) “SITE-SPECIFIC RECOMBINATION AND ITS ROLE IN THE LIFE CYCLE OF BACTERIOPHAGE P1,” Cold Spring Harbor Symp. Quant. Biol. 45:297-309; Hoess, R. et al. (1982) “P1 SITE-SPECIFIC RECOMBINATION: NUCLEOTIDE SEQUENCE OF THE RECOMBINING SITES,” Proc. Natl. Acad. Sci. (U.S.A.) 79:3398-3402; Abremski, K. et al. (1983) “STUDIES ON THE PROPERTIES OF P1 SITE-SPECIFIC RECOMBINATION: EVIDENCE FOR TOPOLOGICALLY UNLINKED PRODUCTS FOLLOWING RECOMBINATION,” Cell 32:1301-1311; Abremski, K. et al. (1984) “BACTERIOPHAGE P1 SITE-SPECIFIC RECOMBINATION: PURIFICATION AND PROPERTIES OF THE CRE RECOMBINASE PROTEIN,” J. Molec. Biol. 259:1509-1514; Hamilton, D. L. et al. (1984) “SITE-SPECIFIC RECOMBINATION BY THE BACTERIOPHAGE P1 LOXP-CRE SYSTEM,” J. Molec. Biol. 178:481-486; Hoess, R. et al. (1984) “INTERACTION OF THE BACTERIOPHAGE P1 RECOMBINASE CRE WITH THE RECOMBINING SITE LOXP,” Proc. Natl. Acad. Sci. (U.S.A.) 81:1026-1209; Hoess, R. et al., (1984) “THE NATURE OF THE INTERACTION OF THE P1 RECOMBINASE CRE WITH THE RECOMBINING SITE LOXP,” Cold Spring Harbor. Symp. Quant. Biol. 49:761-768; Abremski, K. et al. (1986) “BACTERIOPHAGE P1 CRE-LOXP SITE-SPECIFIC RECOMBINATION: SITE-SPECIFIC DNA TOPOISOMERASE ACTIVITY OF THE CRE RECOMBINATION PROTEIN,” J. Biol. Chem. 261:391-396; Sauer, B. (1987) “FUNCTIONAL EXPRESSION OF THE CRE-LOX SITE-SPECIFIC RECOMBINATION SYSTEM IN THE YEAST Saccharomyces cerevisiae,” Molec. Cell. Biol. 7:2087-2096; Abremski, K. et al. (1988) “PROPERTIES OF A MUTANT CRE PROTEIN THAT ALTERS THE TOPOLOGICAL LINKAGE OF RECOMBINATION PRODUCTS,” J. Molec. Biol. 202:59-66; Sauer, B. et al. (1988) “SITE-SPECIFIC DNA RECOMBINATION IN MAMMALIAN CELLS BY THE CRE RECOMBINASE OF BACTERIOPHAGE P1,” Proc. Natl. Acad. Sci. (U.S.A.) 85:5166-5170 (1988); Palazzolo, M. J. et al. (1990) “PHAGE LAMBDA CDNA CLONING VECTORS FOR SUBTRACTIVE HYBRIDIZATION, FUSION-PROTEIN SYNTHESIS AND CRE-LOXP AUTOMATIC PLASMID SUBCLONING,” Gene 88:25-36; Sternberg, N. et al. (1990) “Bacteriophage P1 cloning system for the isolation, amplification, and recovery of DNA fragments as large as 100 kilobase pairs,” Proc. Natl. Acad. Sci. (U.S.A.) 87:103-107; see also U.S. Pat. Nos. 6,448,017; 6,261,808; 6,218,152; 5,834,202; 5,733,733; 5,614,389; 5,591,609; 5,354,668; and 4,959,317).

In a preferred aspect of the invention, at least one of the first lambdoid phage and the second lambdoid phage are constructed by the use of in vivo homologous recombination between a “starter” phage and “donor” nucleic molecules (i.e., molecules comprising linear double stranded or single stranded nucleic acid molecules), in an E. coli recombineering host cell, to create a lambdoid phage comprising a display fusion protein or to introduce changes into a display fusion protein (Oppenheim, A. B. et al. (2004) “IN VIVO RECOMBINEERING OF BACTERIOPHAGE LAMBDA BY PCR FRAGMENTS AND SINGLE-STRAND OLIGONUCLEOTIDES,” Virology 319(2): 185-189). In VIVO homologous recombination is preferably accomplished using at least one or more of the Red functions of bacteriophage lambda. The “Red” functions of bacteriophage lambda, as used herein, refers to the Exo, Beta and Gam proteins of bacteriophage lambda, and to the genes encoding these proteins.

It is recognized that genes encoding Exo, Beta, and Gam may be considerably mutated without materially altering their function. Thus, the invention encompasses the use of any mutated or variant forms of the Red functions that maintain activity in effecting homologous recombination. For example, because the genetic code is degenerate, the genes encoding the Red functions may contain mutated codons that encode the same amino acids as in the unmutated genes. Additionally, even where an amino acid mutation is introduced, the mutation may be a conservative or a non-conservative amino acid substitution that does not critically affect the relevant Red function. Additionally, mutations may be insertions or deletions that do not critically affect the relevant Red function.

The donor nucleic acid molecules will be linear double-stranded or single-stranded molecules that comprise a display protein homologous portion. The term “display protein homologous portion,” as used herein, refers to a portion of the donor nucleic acid molecule that is sufficiently homologous to a portion of the target phage DNA encoding a display protein, or adjacent to DNA sequence encoding a display protein, such that homologous recombination will result in a change in at least one display protein, wherein a target molecule or a target-binder molecule will be incorporated into the display protein, or wherein a previously incorporated target molecule or target-binder molecule will be altered.

In one embodiment, when linear double stranded donor molecules are to be employed, the E. coli recombineering host cells comprise at least the Exo and Beta lambda Red functions, preferably under the control of one or more de-repressible promoters. As used herein, a “de-repressible promoter” refers to a promoter that is substantially less active when bound by a repressor. By regulating the binding of the repressor, such as by changing the environment, the repressor is released from the de-repressible promoter, and transcription increases. As used herein, a de-repressible promoter does not require an activator for transcription. One specific, non-limiting example is the lambda p_{L} promoter, which is regulated by the lambda repressor c_{I}, but which is not activated by an activator. Increased temperature inactivates the temperature-sensitive repressor c_{I}, allowing genes that are operably linked to the p_{L} promoter to be expressed at increased levels. One of skill in the art can readily identify a de-repressible promoter.

In a preferred embodiment, when linear double stranded donor nucleic acid molecules are to be employed, the E. coli recombineering host cells comprise the Exo, Beta, and Gam lambda Red functions, preferably under the control of one or more de-repressible promoters. In another embodiment of the invention, when single-stranded nucleic acid molecules are to be employed, the recombineering host cells comprise at least the Beta function, preferably under the control of a de-repressible promoter. The method preferably comprises the steps of infecting the recombineering host cells with the starter phage, inducing the de-repressible promoter(s) to induce the relevant Exo, Beta and Gam functions, introducing donor molecules that have homology to an insertion site in a display protein or a display fusion protein into the recombineering host cells, incubating the transformed cells to allow completion of the lytic phase by the modified starter phage, and harvesting the resulting first or second lambdoid phage resulting from the recombinantion of the “starter” phage and “donor” molecule.

Thus, in one embodiment, the invention relates to a method of modifying a display protein or a display fusion protein of a lambdoid phage comprising the steps of: (a) providing recombineering host cells; (b) infecting the recombineering host cells with a target lambdoid phage having at least one Red function; (c) inducing the de-repressible promoter to express the Red function; (d) transforming the recombineering host cells with donor nucleic acid molecules comprising a display protein homologous portion; (e) preparing a phage lysate from transformed cells to obtain target lambdoid phage having modified display proteins or display fusion proteins. In vivo recombineering using lambda is discussed by Oppenheim, A. B. et al. (2004) (“IN VIVO RECOMBINEERING OF BACTERIOPHAGE LAMBDA BY PCR FRAGMENTS AND SINGLE-STRAND OLIGONUCLEOTIDES,” Virology 319(2): 185-189).

Donor nucleic acid molecules may be introduced or transformed into the host recombineering cells using any techniques known in the art including, for example, electroporation, calcium phosphate-DNA co-precipitation, DEAE-dextran-mediated transformation, polybrene-mediated transformation, microinjection, liposome fusion, lipofection, protoplast fusion, inactivated adenovirus-mediated transfer, HVJ-liposome mediated transfer, and biolistics. As used herein, the word “transformed” refers to any method of introduction of the donor molecules into the recombineering host cells. In a preferred embodiment, donor molecules are transformed into the host recombineering host cells using electroporation. Various methods and apparatuses useful for electroporation are described in: U.S. Pat. Nos. 4,695,547; 4,764,473; 4,946,793; 4,906,576; 4,923,814; and 4,849,089, all of which are herein incorporated by reference.

In certain embodiments of the invention, particularly when single-stranded donor molecules are employed, recombineering host cells will contain mutations in the methyl-directed mismatch repair (MMR) system, including mutations in the mutH, mutL, mutS, uvrD, and dam genes that eliminate or substantially reduce the mismatch repair functions of these genes. Advantages of recombineering host cells having defects in the MMR genes are described in Constantino, N. et al. (2003) “ENHANCED LEVELS OF λ RED-MEDIATED RECOMBINANTS IN MISMATCH REPAIR MUTANTS,” Proc. Natl. Acad. Sci. USA 100:15748-15753).

In certain embodiments of the invention, the lambdoid strains employed may comprise long-circulating strains that allow for a longer period of circulation in vivo for lambdoid strains that are employed as therapeutic or diagnostic agents as described in Merril, C. et al. (1996) (“LONG-CIRCULATING BACTERIOPHAGE AS ANTIBACTERIAL AGENTS,” Proc. Natl. Acad. Sci. USA 93:3188-3192). Particularly preferred lambdoid strains comprise mutations in the E gene that allow for a longer in vitro half-life.

### Applications of the Invention

Applications of the invention include a wide a variety of procedures to identify binding protein pairs or to refine or optimize binding pairs. Preferred examples of various applications of the invention are listed below.

### Proteome Panning to Identify Binding Pairs

The methods of the invention may be used to identify protein binding pairs using unknown target molecules. In this aspect of the invention, a first population of lambda phage particles displaying a first library of peptides and polypeptides and a second population of lambda phage particles displaying a second library of peptides and polypeptides are mixed added to host cells. Host cells that are co-infected a lambda phage particle of the first population and a lambda phage particle of the second population are identified, and the proteins displayed on the lambda phage particles that have co-infected the host cell are identified as binding pairs. In a preferred embodiment of the embodiment of the invention either or both of the first library and the second library comprises greater than 10⁶ members, preferably greater than 10⁷ members.

### Proteome Panning to Identify Binding Partners for a Particular Ligand.

The methods of the invention may be used to identify binding partners for a particular ligand. In this aspect of the invention, a first population of lambda phage particles displaying a particular peptide or polypeptide (i.e., the target ligand) and a second population of lambda phage particles displaying a library of peptides and polypeptides are mixed and added to host cells. Host cells that are co-infected with a lambda phage particle of the first population and a lambda phage particle of the second population are identified, and the proteins displayed on the lambda phage particle of the second population is identified as binding partner for the target ligand.

### Directed Evolution

The methods of the invention may be used for the directed evolution of an amino acid sequence with regard to the binding affinity of the amino acid sequence for a particular ligand. The term “directed evolution,” as used herein, refers to the process of bringing forth a novel amino acid sequence from a starting amino acid sequence by randomly or selectively mutating the amino acid sequence and then imposing rationally designed selection conditions and pressures. For example, an amino acid sequence would be randomly or selectively mutated and then selected for some aspect of binding affinity for a particular ligand using the methods of the instant invention. Selection pressures that might be applied include, for example, the following: selection for higher binding affinity; selection for higher binding affinity under particular conditions such as pH, temperature etc.; selection of retained binding affinity in the presence of an alternative ligand; selection for lower binding affinity; selection for lower binding affinity under particular conditions such as pH, temperature etc.; selection for decreased binding affinity in the presence of an alternative ligand, and selection for greater or decreased specificity in binding under various conditions.

### Isolation of Cell Reactive Antibodies

The methods of the invention may be used to identify cell reactive antibodies and the corresponding epitopes. Such cell reactive antibodies may be employed to identify a wide variety of cell types (e.g., cancer cells, hormone (e.g., insulin, etc.) producing cells, cells whose presence is characteristic of a disease state (e.g., Alzheimer's Disease, etc.). In this application of the invention, a first population of phage particles that display a single chain FV (scFV) antibody library derived from naïve animals or from animals immunized with whole cells of a desired (e.g., cancer) cell type X is constructed (Popkov, M. et al. (2004) “ISOLATION OF HUMAN PROSTATE CANCER CELL REACTIVE ANTIBODIES USING PHAGE DISPLAY TECHNOLOGY,” J. Immunol. Methods 291(1-2): 137-51). A second population of phage particles that display the expressed proteins of a cDNA library derived from cell type X is also constructed. Preferably, the phage display antibody library is then subjected to a negative selection pre-screen to remove antibodies that react with non-cancerous cells. For example, the phage display antibody library may be pre-screened by contacting the library with non-cancerous cells related to the cancer cell type X to remove antibodies that are not specific for the cancer cells. The two phage populations are then mixed, and the mixture is then assayed for phage complex formation, wherein phage complex formation is indicative of a binding interaction between an scFV antibody and a corresponding epitope of a protein expressed from a cDNA molecule derived from the cancer cell type X. Significantly, the mixed populations of phage may be independently selected from: (1) purified clones (i.e., a population composed of genetically identical phages), (2) mixtures of related purified clones (i.e., a population composed of multiple different but related species of phages, such as a mutagenized preparation derived from a population of genetically identical phages), or (3) libraries of genetically different phages. The process of mixing the two populations of phage can be repeated to obtain increasingly strong-interacting phage species.

### Screening for Protein-Binding Modulators

The methods of the invention may be used to screen for modulators of protein/protein binding interactions, referred to herein as “protein-binding modulators”. The method comprises the steps of: (a) forming a reaction mixture comprising a first lambdoid phage that displays a target molecule and a second lambdoid phage that displays a target-binder molecule, in the presence and absence of a test modulator, under conditions permissive for a binding interaction between said target molecule and said target-binder molecule; (b) contacting said mixture with host cells under conditions permissive for lambdoid phage infection of said host cells; and (c) assaying said host cells for co-infection by said first lambdoid phage and said second lambdoid phage and observing the effect of the test modulator on the number of co-infections, wherein co-infection is indicative of a binding interaction between said target molecule and said target-binder-molecule, and wherein said test modulator is identified as a protein-binding modulator if the number of co-infections in the presence of the test modulator is greater or less than the number of co-infections in the absence of the test modulator. It is contemplated that a protein-binding modulator may be a binding potentiator, i.e. an agent that positively affects the binding of a target molecule and a target-binder molecule, or binding inhibitor, i.e. an agent that negatively affects the binding of a target molecule and a target-binder molecule. It is contemplated that test modulators may comprise, for example, peptides or polypeptides, peptide mimetics, organic molecules, nucleic acid molecules etc. In a preferred embodiment of the invention, the methods of the invention are employed to screen for putative therapeutic agents that are binding inhibitors.

Having now generally described the invention, the same will be more readily understood through reference to the following examples, which are provided by way of illustration and are not intended to be limiting of the present invention unless specified.

### Example 1

### High-Density Display of Proteins on Bacteriophage Lambda

The present invention is illustrated by reference to a cloning strategy based on first inserting DNA encoding peptide-protein into a high copy donor plasmid vector and then transferring this genetic information into a recipient lambda genome, using the high-efficiency lox-Cre recombination system in vivo.

### Materials

E. coli strain BM25.8 {supE thiΔ (lac-proAB) [F′traD36proA+B+lacIqZΔM15]imm434 (kanr) P1 (Cmr) hsdR (r−m+)} (Novagen, Madison, Wis.) is used as the Cre+host for in vivo recombination. E. coli strain TG1 (supEΔ(hsdM-mcrB)5(rk-mk-McrB-)thiΔ(lac-proAB) [F′traD36, LacIqΔ(lacZ)M15]) is used as the Cre-host for titering phage lysates and amplification of phages. λDam imm21 nin5 (Sternberg, N. et al. (1995) “DISPLAY OF PEPTIDES AND PROTEINS ON THE SURFACE OF BACTERIOPHAGE LAMBDA,” Proc. Natl. Acad. Sci. USA 92(5):1609-1613) is used for constructing DL1. Collagenase is obtained from Roche Diagnostics, Germany. Anti-c-myc mAb, 9E10 is produced using hybridoma obtained from ATCC, Manassas, Va. Anti-p24 mAb, H23 is produced in-house and its epitope mapped (amino acid residues 56-66 of HIV-1 p24) using a phage display-based gene-fragment library (Gupta, S. et al. (2001) “MAPPING OF HIV-1 GAG EPITOPES RECOGNIZED BY POLYCLONAL ANTIBODIES USING GENE-FRAGMENT PHAGE DISPLAY SYSTEM,” Prep Biochem Biotechnol. 31(2):185-200). GST-c-myc is produced in E. coli and purified to homogeneity by affinity chromatography. mAbs to PE are raised by immunizing mice with a derivative of PE-38 carrying mutation in the active site. The human sera are anonymous samples obtained from patients undergoing immunotoxin therapy and collected after informed consent. HRP-conjugated antibodies may be obtained from Jackson ImmunoResearch Laboratories (West Grove, Pa.).

### Construction of Donor Plasmid Vectors

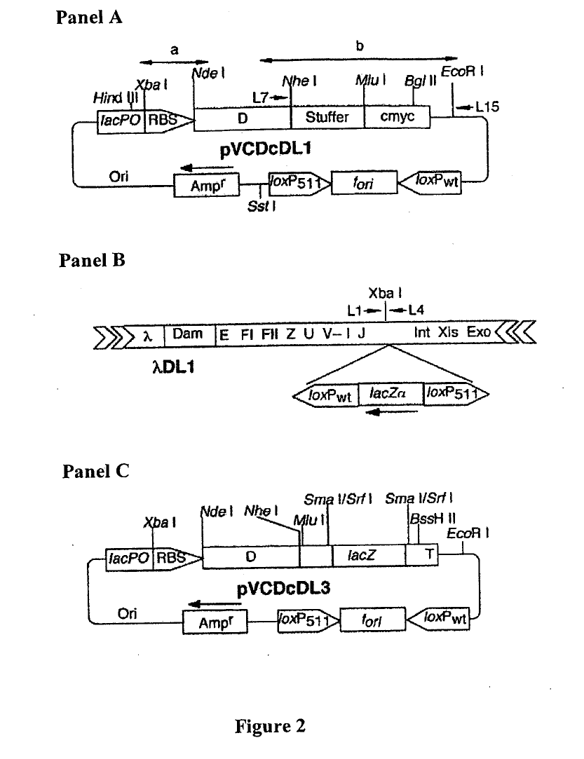

The donor plasmid vector, pVCDcDL1, is assembled by ligating the following three segments of DNA bearing compatible ends. One segment is prepared by PCR-based amplification of the lambda D gene to create a HindIII site before the Shine-Dalgarno sequence and to incorporate after the last codon of D gene, a sequence encoding spacer (PGGSG) (SEQ ID NO:1), followed by a collagenase cleavage site (PVGP), NheI site, ten codons of a stuffer sequence, codons for decapeptide tag, c-myc, stop codon, and SalI and EcoRI restriction sites. The assembled PCR product is digested with HindIII and EcoRI to obtain a 475 bp fragment. The second segment is also assembled by PCR and contained the origin of replication of filamentous phage (fₒᵣᵢ) flanked by the sequence for restriction site SstI and loxP₅₁₁ (Hoess, R. H. et al. (1986) “THE ROLE OF THE LOXP SPACER REGION IN P1 SITE-SPECIFIC RECOMBINATION,” Nucleic Acids Res. 14(5):2287-2300) on one end and the sequence for loxP_{wt} and an EcoRI restriction site on the other end. The product is digested with SstI and EcoRI to obtain a 515 bp fragment. The third segment formed the backbone of the plasmid vector. For this, an SstI restriction site is created by site-directed mutagenesis in pUC119 upstream of the -lactamase gene to produce a plasmid pUCSSt. pUCSSt is digested with HindIII and SstI and dephosphorylated to obtain a 2.5 kb fragment. pVCDcDL1 (GenBank Accession No. AY10049), is obtained from ligation of the three fragments, and sequenced between HindIII and SstI sites using the dideoxy chain termination method. pVCDcDL3 (GenBank Accession No. AY190304) is constructed by cloning a cassette encoding the lac promoter, RBS and the first 145 codons of lacZ flanked by SmaI/SrfI sites, as NheI-EcoRI insert in pVCDcDL1 (FIG. 2, Panel C).

### Construction of Recipient Lambda Vector

A DNA segment comprising the lac promoter, RBS and first 58 codons of lacZ flanked by sequence for loxP₅₁₁, and lox P_{wt} is assembled by PCR to have XbaI compatible ends and ligated in the unique XbaI site in Dam at map co-ordinate 24508. The ligation mix is then packaged in vitro using the Gigapack II system (Stratagene, La Jolla, Calif.). The phage mixture produced after packaging is plated on lawn cells (E. coli strain TG1). The plaques obtained are analyzed for recombinants by PCR using primers L1 and L4, which flank the XbaI site in lambda (FIG. 2, Panel B). The recombinant obtained is named DL1.

### Generation of Lambda Cointegrates and Phage Production

BM 25.8 cells (Cre⁺) and TG1 cells (Cre⁻) transformed with donor plasmid (carrying foreign DNA) are grown to A_{600 nm}˜0.3 in LBAmp (LB medium containing ampicillin at 100 μg/ml) at 37° C. Cells (1×10⁸) are harvested and suspended in 100 μl of DL1 phage lysate at an MOI of 1.0. After incubation at 37° C. for ten minutes, the sample is diluted in 1 ml of LBAmp containing MgCl₂ (10 mM) and grown at 37° C. with shaking for three hours for lysis. For large-scale recombination, the number of cells and the volume of DL1 are increased proportionately to maintain an MOI of 1.0. The cell-free supernatant is used to infect an exponential phase culture of TG1 and Amp^{r} colonies obtained. These Amp^{r} colonies are immune to superinfection and carry the phage as plasmid cointegrates. The Amp^{r} colony containing the lambda cointegrate is grown in LBAmp at 37° C. for four hours. Lambda phage are spontaneously induced in these cultures and result in complete lysis. This cell-free supernatant is then used to infect TG1 cells to obtain plaques. Phage obtained from single plaques are amplified by the liquid lysis method at an MOI of 0.01 to obtain lysate with a titre of 5×10⁹ pfu per ml. These phage are further amplified by the liquid lysis method and purified by PEG-NaCl precipitation and differential sedimentation.

### Construction of Lambda and M13 Vectors for Display of Various Fragments of p24

DNA sequences encoding different fragments of HIV capsid protein p24 are amplified from pVCp24210 (Gupta, S. et al. (2000) “GAG-DERIVED PROTEINS OF HIV-1 ISOLATES FROM INDIAN PATIENTS: CLONING, EXPRESSION, AND PURIFICATION OF P24OF B- AND C-SUBTYPES,” Protein Expr Purif. 19(3):321-328) and cloned between NheI-MluI sites to replace the stuffer fragment in pVCDcDL1 and create donor plasmids pVCDc(p241)DL1/pVCDc(p246)DL1 and pVCDc(p24)DL1. E. coli strain BM25.8 is transformed with each plasmid and recombination carried out by infecting cultures of each transformant with DL1 phage to obtain DCO cointegrates of Dc(p241)DL1, Dc(p246)DL1 and Dc(p24)DL1 as described above.

DNA encoding different p24 fragments are also cloned as NheI-MluI inserts into phagemid gIII display vector, pVC3TA726 (Sampath, A. et al. (1997) “VERSATILE VECTORS FOR DIRECT CLONING AND LIGATION-INDEPENDENT CLONING OF PCR-AMPLIFIED FRAGMENTS FOR SURFACE DISPLAY ON FILAMENTOUS BACTERIOPHAGES,” Gene 190(1):5-10), and a similar phagemid gVIII display vector, pVCp240518426 to obtain various phagemid constructs to produce phage displaying protein fused to gIII and gVIII p of M13, respectively. The M13 phage displaying proteins are produced by using VCS M13 as described (Kushwaha, A. et al. (1994) “CONSTRUCTION AND CHARACTERIZATION OF M13 BACTERIOPHAGES DISPLAYING FUNCTIONAL IGG-BINDING DOMAINS OF STAPHYLOCOCCAL PROTEIN A,” Gene 30;151(1-2):45-51). The lambda and M13 phage are purified from cell-free supernatant by PEG precipitation followed by ultracentrifugation.

### Construction of Pseudomonas Exotoxin (PE) Gene-Fragment Library in and M13 Vectors

Random fragments (50-200 bp) of DNA encoding PE-38, a 38 kDa fragment of PE (Debinski, W. et al. (1992) “MONOVALENT IMMUNOTOXIN CONTAINING TRUNCATED FORM OF PSEUDOMONAS EXOTOXIN AS POTENT ANTITUMOR AGENT,” Cancer Res. 52(19):5379-5385) are produced by DNase I digestion and ligated as blunt-ended fragments (1 g) in SmaI (CCCGGG) (SEQ ID NO:2)-digested pVCDcDL3 (500 ng) in the presence of restriction enzyme SrfI (GCCCGGGC) (SEQ ID NO:3) using previously described protocols (Gupta, S. et al. (1999) “SIMPLIFIED GENE-FRAGMENT PHAGE DISPLAY SYSTEM FOR EPITOPE MAPPING,” Biotechniques. 27(2):328-30, 332-334). The ligation mix is electroporated into BM25.8 cells and plated on 150 mm LBAmpGlu (LBAmp medium containing 1% glucose) plates to obtain 5×10⁶ independent clones. The transformants are scraped and cell suspension stored at −70° C. An aliquot of stored cell suspension (1×10⁸ cells) of the library is grown in 10 ml of LBAmpGlu to an A600 of 0.3. The cells are harvested and suspended in 1 ml of DL1 phage lysate at an MOI of 1.0. After incubation at 37° C. for ten minutes, the samples are diluted in 10 ml of LBAmp containing MgCl₂ (10 mM) and grown at 37° C. with shaking for three to four hours until cell lysis. The cell-free supernatant (10 ml) is used to infect an exponential phase culture of TG1 cells (10 ml) at 37° C. for ten minutes and the cell suspension is plated on 20 LBAmpGlu 150 mm plates. The Ampr colonies harboring cointegrates are scraped and stored at −70° C. Cells (1×10⁹) harboring cointegrates are diluted into 50 ml of LBAmp medium and grown at 37° C. for eight hours to produce phage particles. The cell-free supernatant containing phage particles is directly used for affinity selection. PE-derived 50-200 bp DNA fragments are also ligated to SmaI-digested phagemid-based gIIIp display vector, pVCEPI13426, to obtain the gene-fragment library in M13. A library of 6×10⁶ independent clones is obtained in TG1 cells and used to produce M13 phage displaying peptides as described Kushwaha, A. et al. (1994) (“CONSTRUCTION AND CHARACTERIZATION OF M13 BACTERIOPHAGES DISPLAYING FUNCTIONAL IGG-BINDING DOMAINS OF STAPHYLOCOCCAL PROTEIN A,” Gene 30;151(1-2):45-51).

Construction of scFv Displaying Lambda Phage

DNA encoding the scFv fragment of the anti-mesothelin antibody, SS1 is PCR amplified using pPSC7-1-1 (Chowdhury, P. S. et al. (1999) “IMPROVING ANTIBODY AFFINITY BY MIMICKING SOMATIC HYPERMUTATION IN VITRO,” Nature Biotechnol. 17:568-572) as template and cloned as an NheI-MluI insert in pVCDcDL1, to obtain donor plasmid pVCDcSS1DL1. BM25.8 cells are transformed with pVCDcSS1DL1 and recombination performed using DL1 as described above to isolate a clone harboring DCO cointegrate, DcSS1DL1. A single colony harboring DCO cointegrate is grown in LBAmp at 37° C. for four to six hours for lysis to occur. The supernatant is used to grow more phage by the liquid lysis method in LB medium by infecting TG1 cells at MOI 0.01. Phage from cell-free supernatant are purified by PEG-NaCl precipitation and differential sedimentation.

### Estimation of Phage Binding and Affinity Selection of Binders by Bio-Panning

To check the presence of binder phage, wells of microtiter plates (Maxisorp, Nunc, Rochester, N.Y.) are coated with 1:1000 dilution of ascitic fluid of anti-c-myc mAb 9E10 and phage lysate is added to the coated wells (Gupta, S. et al. (1999) “SIMPLIFIED GENE-FRAGMENT PHAGE DISPLAY SYSTEM FOR EPITOPE MAPPING,” Biotechniques. 27(2):328-30, 332-334) and incubated for one hour at 37° C. The unbound phage are removed by washing. To assay the captured lambda phage, 0.3 ml of exponential phase TG1 cells are added to each well and incubated for ten minutes at 37° C. Cells are then removed and serial dilutions plated to determine phage-infected cells as pfu and cfu. The pfu and cfu indicate the number of phage bound to the coated wells. For panning of the PE gene-fragment library on mAb, wells are first coated with goat anti-mouse IgG (Fc fragment-specific) antibody followed by 1:100 dilution of anti-PE mAb culture supernatant (Test wells) or buffer (Control wells). For panning of the PE gene-fragment library on human serum, wells are coated with goat anti-human (IgG+IgM, Fc fragment-specific) antibody followed by 1:100 dilution of serum from patients treated with PE-based immunotoxins (Test wells) or pre-treatment serum of patients (Control wells). Phage lysate (1×10⁸ phages per well for lambda library and 1×10¹⁰ phage per well for M13 library) is added to each well, incubated at 37° C. for one hour and unbound phages removed by washing. For the M13 library, the captured phage are eluted using low-pH buffer (Gupta, S. et al. (1999) “SIMPLIFIED GENE-FRAGMENT PHAGE DISPLAY SYSTEM FOR EPITOPE MAPPING,” Biotechniques. 27(2):328-30, 332-334) and titrated on TG1 as cfu. In the case of lambda phage, one unit of collagenase in 0.1 ml of phosphate buffer (20 mM, pH 7.4) is added to each well for ten minutes at room temperature. The released phages are titrated on TGI to obtain Ampr colonies. Individual Ampr colonies are grown and phage particles produced as described previously by infecting with helper phage for M13 clones (Kushwaha, A. et al. (1994) “CONSTRUCTION AND CHARACTERIZATION OF M13 BACTERIOPHAGES DISPLAYING FUNCTIONAL IGG-BINDING DOMAINS OF STAPHYLOCOCCAL PROTEIN A,” Gene 30;151(1-2):45-51) and by growing colonies in LBAmp medium till complete cell lysis for lambda phage clones. The cell-free supernatants are subsequently used for ELISA.

### Western Blot Analysis and ELISA of Phage

For Western blots, purified phage are electrophoresed under reducing conditions on 0.1% (w/v) SDS/10% or 12.5% (w/v) PAG followed by electroblotting onto PVDF membrane (Immobilon, Millipore, Bedford, Mass.). Fusion proteins are detected with 1:1000 dilution of ascitic fluid of anti-c-myc mAb, 9E10/anti-p24 mAb, H23 followed by horse radish peroxidase (HRP)-conjugated goat anti-mouse IgG (H+L) antibody. For ELISA, wells of Maxisorp plates (Nunc, Rochester, N.Y.) are coated with 1:1000 dilution of ascitic fluid of mAb 9E10/H23 and purified phage are added to the coated wells. The bound phages are detected with rabbit anti-lambda polyclonal serum or rabbit anti-M13 polyclonal serum followed by HRP-conjugated goat anti-rabbit IgG (H+L) antibody. Binding of phages produced by individual clones selected in bio-panning is tested in ELISA. For this, wells are coated with 1:1000 dilution of rabbit anti-lambda polyclonal serum or rabbit anti-M13 polyclonal serum and corresponding phages are added to the coated wells. After removing unbound phage, 1:100 dilution of anti-PE mAb (culture supernatant) or serum from patients treated with PE-based immunotoxins is added. The bound phage are detected with HRP-conjugated goat anti-mouse IgG (H+L) antibody or HRP-conjugated goat anti-human (IgG+IgM) antibody. For ELISA of phages displaying SS1 scFv, microtiter wells are coated with 100 ng of recombinant mesothelin (Chowdhury, P. S. et al. (1999) “IMPROVING ANTIBODY AFFINITY BY MIMICKING SOMATIC HYPERMUTATION IN VITRO,” Nature Biotechnol. 17:568-572). After blocking the unoccupied sites with 2% non-fat dry milk, purified lambda phage are added to the coated wells and incubated at 37° C. for one hour. The unbound phage are removed by washing and the bound phage detected with rabbit anti-lambda polyclonal serum followed by HRP-conjugated goat anti-rabbit IgG (H+L) antibody.

### Results

Cloning into Lambda Display Vector by in vivo Recombination

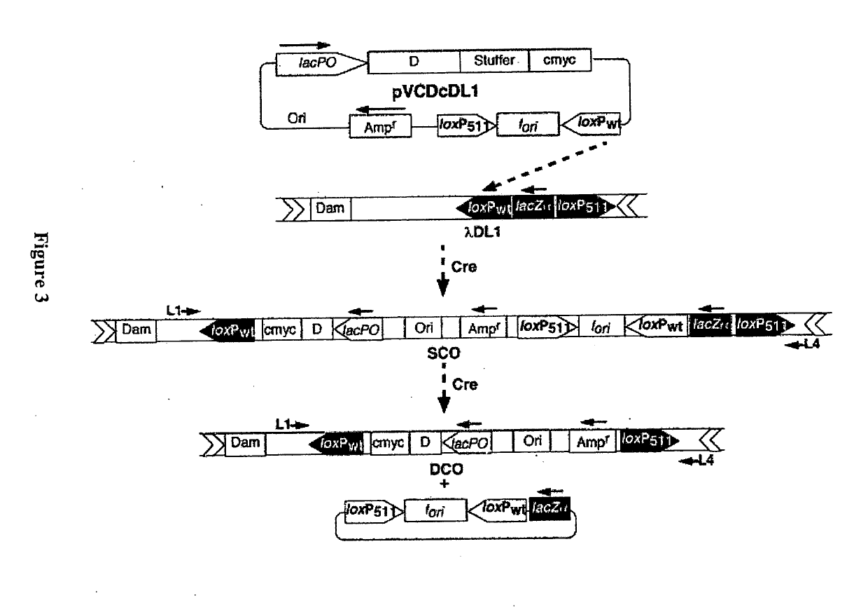

DNA encoding the peptide-protein is introduced into a high copy donor plasmid vector, pVCDcDL1 (FIG. 2, Panel A), and then transferred to recipient lambda genome, DL1 (FIG. 2, Panel B), by the high-efficiency lox-Cre recombination system in vivo (FIG. 3). The plasmid pVCDcDL1 contains a sequence encoding gpD of λ, followed by a PGGSG (SEQ ID NO:1) spacer, a collagenase site, an NheI site, a stuffer segment, a MluI site and a c-myc tag, under the control of the lac promoter (lacPO). Cloning of DNA sequences as NheI-MluI inserts in place of the stuffer allows for formation of a D fusion protein with a collagenase site between D and the foreign protein and c-myc tag at the C terminus. The vector also contains the M13 phage origin of replication (fₒᵣᵢ), flanked by loxP_{wt} and loxP₅₁₁ recombination sequences. The recipient lambda vector, DL1, contains a lacZ fragment flanked by loxP_{wt}, and loxP₅₁₁ recombination sequences at the unique XbaI site present in the lambda genome. The lox sequences in the donor plasmid are in the reverse orientation to that in the recipient lambda genome (FIG. 2, Panel B). When Escherichia coli expressing Cre recombinase (Cre⁺ host) are transformed with the donor plasmid and then infected with DL1, recombination occurs at the compatible lox sites in the two vectors, resulting in integration of the plasmid DNA into the lambda DNA (FIG. 3). Note that Cre-mediated recombination occurs between two loxP_{wt} sites or between two loxP₅₁₁ sites, and not between a loxP_{wt} and a loxP₅₁₁ site (Hoess, R. H. et al. (1986) “THE ROLE OF THE LOXP SPACER REGION IN P1 SITE-SPECIFIC RECOMBINATION,” Nucleic Acids Res. 14(5):2287-2300). Hence, plasmid and lambda DNA crossing over occurs only in trans and results in the formation of a cointegrate. Additionally, due to opposite orientation of the lox sites in the plasmid and lambda, the recombination leads to integration of the entire plasmid DNA into the lambda DNA. The first crossover event (intermolecular) results in the formation of single crossover (SCO) cointegrate that contains the complete donor plasmid integrated in the lambda genome. A second crossover event (intramolecular) at the other pair of compatible lox sites results in the formation of a double crossover (DCO) cointegrate and excision of the lacZα fragment and fₒᵣᵢ sequence (FIG. 3). Thus, the DNA encoding the foreign peptide/protein fused to gpD for display on the lambda phage surface becomes part of the lambda genome. The lambda also acquires the β-lactamase selection marker of the plasmid.

Based upon this strategy, BM25.8 (Cre⁺ host) and TG1 (Cre⁻ host) are transformed with the donor plasmid, pVCDcDL1, and then infected with recipient lambda phage, DL1. The cultures are grown in ampicillin-containing medium until complete cell lysis. The cell-free lysate obtained after the recombination event is used to infect Cre⁻ cells, and the cells are plated to determine plaque-forming units (pfu) and colony-forming units (cfu) (on ampicillin-containing medium). The number of pfu is the same in the lysate obtained from Cre⁺ and Cre⁻ hosts, indicating similar amounts of phage production in both hosts. However, only the lysate from Cre⁺ host is able to transduce Amp^{r} colonies in E. coli. This result indicates that the plasmid integrates into lambda DNA only in the presence of Cre protein and confers ampicillin resistance to cells harboring this lambda cointegrate as an extra chromosomal lysogen driven by the plasmid replicon. The lysate from Cre⁺ host contains three phage species: parental recipient lambda, SCO cointegrate (DcDL1: SCO) and DCO cointegrate (DcDL1: DCO). Plating on ampicillin-containing medium selects for cointegrates and eliminates parental phage. To check for the presence of plasmid sequence in lambda genome, the Amp^{r} colonies are analyzed by PCR using primers L1 and L4 (FIG. 3) that flank the lox sequences in lambda. Agarose gel electrophoresis of amplified products shows that all the colonies analyzed harbored cointegrates and the ratio of SCO to DCO cointegrates is 1:3. DcDL1: SCO and DcDL1: DCO harboring clones are grown in ampicillin-containing medium wherein there is spontaneous phage production leading to cell lysis. The cell-free lysates are tested for phage titre and presence of gpD-c-myc protein on the phage surface. Both SCO and DCO harboring cells produced the same number of pfu. To test the stability of phage particles, the lysates are incubated in EDTA-containing buffer and then re-titrated to determine the number of viable phages. No difference in pfu before and after incubation in EDTA is observed for lysates obtained from SCO and DCO clones, indicating that the phages produced are resistant to EDTA and all 405 copies of gpD (either as gpD or gpD fusion protein) are present on every phage particle (Georgopoulos et al., In: R. W. Hendrix, J. W. Roberts, F. W. Stahl and R. A. Weisberg, Editors, Lambda II, Cold Spring Harbor Laboratory Press, Cold Spring Harbor, N.Y. (1983)). The phage particles are tested for display of c-myc peptide as gpD fusion. Both types of phage displayed the same amount of c-myc peptide as revealed by equal recovery of phages (˜2% of phages added) following bio-panning in anti-c-myc (mAb 9E10) coated wells. This recovery is at least 200-fold higher than that obtained for DL1 phage (that does not display gpD-c-myc). Western blot analysis with mAb 9E10 shows that phages purified from lysate of both SCO and DCO clones shows a band of ˜16 kDa, with an intensity corresponding to ˜400 copies of fusion protein per phage particle (the number of fusion proteins is calculated by densitometric scanning of the blot using a purified c-myc-containing protein as control). These experiments establish that SCO and DCO phage have similar properties and all amp^{r} transductants resulting from recombination produce functional phage displaying gpD fusion protein.

High Density Display of Peptides and Proteins on Lambda: a Comparison with M13

Display of different size molecules on lambda phage and a comparison with the M13 phage display system in terms of density and functionality of displayed peptides and proteins is carried out using fragments of HIV-1 capsid protein p24. HIV-1 p24 contains two independently folding domains. The first 156 amino acid residues of p24 constitute the N-terminal domain that interacts with host proteins such as cyclophilin, while residues 157-231 constitute the C-terminal domain, which is responsible for oligomerisation of p24 to form the viral capsid. Three fragments of p24 encompassing residues 1-72 (p241), 1-156 (p246, N-terminal domain of p24) and 1-231 (p24, full-length protein) are displayed as C-terminal fusions with gpD on lambda, and as N-terminal fusions with gVIIIp and gIIIp on M13 using phagemid-based vectors. All the fusion proteins contain a c-myc tag at the C terminus of p24 fragment. Phage are prepared for all lambda and M13 clones and purified by polyethylene glycol (PEG) precipitation and ultracentrifugation. The purified phages are then tested for binding to anti-p24 mAb in ELISA and the display of fusion protein on the phage surface is quantified by Western blot using anti-c-myc mAb 9E10. In ELISA, both M13 and lambda phage displaying p24 fragments show dose-dependent binding to mAb H23, which recognizes amino acid residues 56-66 of p24. p241-displaying phage show maximum reactivity followed by p246-displaying and p24-displaying phage. For all of the three displayed molecules, lambda phage showed two to three orders of magnitude better reactivity compared to corresponding M13 phage, indicating higher display of the proteins.

The number of fusion protein molecules displayed per phage particle is quantified by Western blot analysis using mAb 9E10. In the case of lambda phage, an intense band corresponding to the calculated molecular mass is seen for each of the three fusion proteins. The number of fusion protein molecules displayed per phage particle is estimated to be 350 copies of gpD-p241-c-myc (22 kDa), followed by 210 copies of gpD-p246-c-myc (31 kDa) and 154 copies of gpD-p24-c-myc (39 kDa). In the case of M13, the lane corresponding to phage displaying p241 shows only one band having molecular mass (˜13 kDa as gVIIIp fusion and ˜60 kDa as gIIIp fusion) less than calculated for the fusion protein. Since the full-length fusion protein is not visible on the blot, the amount of p241 fusion protein on M13 phage could not be determined. The lane corresponding to M13 phage displaying p246 and p24 shows two major bands in each blot. The band with slower mobility corresponded to the calculated molecular mass of the fusion protein but the second, more intense band, shows mobility similar to that seen in the lane with M13 phage displaying p241, suggesting these to be degradation products that had retained the c-myc epitope. This faster moving band is reactive to mAb 9E10 but not to mAb H23, confirming the loss of amino acids from the N terminus. Densitometric scanning shows that M13 phage displayed less than two copies of the fusion protein per phage particle. The Western blot data obtained with mAb 9E10 correlates well with the ELISA data obtained for reactivity of phage to mAb H23. The full-length p241-gVIIIp/gIIIp fusion protein may be present in extremely low quantities on M13 phage (not detected in Western blot); however, the degradation product that is displayed on the phage surface retained H23 epitope (confirmed by Western blot of phages using H23) resulting in the high reactivity observed in ELISA. This analysis clearly shows that the lambda phage system is capable of displaying proteins of different sizes with large domains in much higher density than the M13 phage system, with less degradation of the fusion protein.

### Display of Disulfide Bond-Containing Proteins

One major application of phage display technology is the identification of protein-protein interaction cascades in which a plethora of protein sequences are displayed on the phage surface, several of which might contain disulfide bonds essential for their function. The single-chain fragment (scFv) of an antibody was used as a fusion partner with gpD to test the display of disulfide-containing proteins in functional form on lambda. An scFv molecule contains two intra-molecular disulfide bonds, which are essential for its correct conformation and activity. Therefore, functional display of scFv as gpD fusion on lambda surface will indicate that disulfide bonds are formed in proteins displayed on lambda.

Mesothelin is a glycoprotein present on the surface of cancer cells and is a promising candidate for targeted therapies. SS1 is a high-affinity variant of anti-mesothelin antibody SS (Chowdhury, P. S. et al. (1998) “ISOLATION OF A HIGH-AFFINITY STABLE SINGLE-CHAIN FV SPECIFIC FOR MESOTHELIN FROM DNA-IMMUNIZED MICE BY PHAGE DISPLAY AND CONSTRUCTION OF A RECOMBINANT IMMUNOTOXIN WITH ANTI-TUMOR ACTIVITY,” Proc. Natl. Acad. Sci. USA 95:669-674; Chowdhury, P. S. et al. (1999) “IMPROVING ANTIBODY AFFINITY BY MIMICKING SOMATIC HYPERMUTATION IN VITRO,” Nature Biotechnol. 17:568-572). Lambda phage displaying SS1 scFv (DcSS1DL1) are produced by recombination as described in Materials and Methods and purified. These phages display SS1 scFv fused at the C terminus of gpD with a c-myc tag at the C terminus of scFv. In ELISA on anti-c-myc-coated plates, the binding of DcSS1DL1 is about 30 times less than that of DcDL1. Thus, DcSS1DL1 displays about 10-15 copies of D-scFv-c-myc fusion protein in comparison to DcDL1 that displayed 400 copies of D-c-myc fusion protein per phage particle. Functionality of SS1 scFv displayed on lambda is checked by binding of phage to the natural ligand of SS1, mesothelin. DcSS1DL1 phage are added to mesothelin-coated wells and captured phage detected using anti-lambda phage polyclonal sera. DcSS1DL1 phage showed specific dose-dependent binding to mesothelin, indicating that the displayed scFv molecules are functional. DL1 and DcDL1 phages that did not display SS1 scFv showed no binding to mesothelin. Further, 5×10⁹ DcSS1DL1 phages give the same binding to mesothelin as 1×10¹¹ M13 phage displaying SS1 scFv fused to gulp, indicating that the number of functional scFv molecules present per lambda particle is several-fold more than per M13 particle. This is confirmed by Western blot analysis using anti-c-myc mAb 9E10. Here, 1×10⁹ DcSS1DL1 phage showed a band corresponding to gpD-scFv-c-myc fusion protein while a same intensity band of scFv-c-myc-gIIIp is seen with 5×10¹⁰ M13 phage displaying SS1scFv fused to gulp. This result establishes that disulfide bond-containing proteins are also displayed in higher numbers on lambda phage as compared to M13 phage.

### Example 2

### Creating Mutations In Lambda Phage Display Proteins With Recombineering

### The Recombineering Process

Recombinogenic engineering methodology, also known as recombineering, utilizes homologous recombination to create targeted changes in lambda DNA (Oppenheim, A. B. et al. (2004) “IN VIVO RECOMBINEERING OF BACTERIOPHAGE LAMBDA BY PCR FRAGMENTS AND SINGLE-STRAND OLIGONUCLEOTIDES,” Virology 319(2):185-189). Recombineering may be employed to create mutations in lambda phage display proteins and display fusion proteins, as defined herein, by targeting DNA fragments or single stranded-oligonucleotides to phage display proteins. In one example, an Escherichia coli cell harboring a defective prophage is infected with the phage to be engineered. The defective prophage carries the pL operon under control of the cI^{ts}857 temperature-sensitive repressor. The lysogen is induced to express the Red functions, the induced cells are made competent for electroporation, and the DNA fragments or single-stranded oligonucleotides are introduced by electroporation. Following electroporation, a phage lysate is made from the electroporation mix.

The strains used for recombineering carry a defective prophage containing the pL operon under control of the temperature-sensitive repressor cI^{ts}857. The genotype of one commonly used strain, DY330, is W3110 ΔlacU169 gal490 pglΔ8 cI^{ts}857 Δ(cro-bioA). Other useful strains are listed in Ellis, H. M. et al. (2001) (“HIGH EFFICIENCY MUTAGENESIS, REPAIR, AND ENGINEERING OF CHROMOSOMAL DNA USING SINGLE-STRANDED OLIGONUCLEOTIDES,” Proc. Natl. Acad. Sci. USA 98:6742-6746) and Yu, D. et al. (2000) (“AN EFFICIENT RECOMBINATION SYSTEM FOR CHROMOSOME ENGINEERING IN Escherichia coli,” Proc. Natl. Acad. Sci. USA 97:5978-5983).

### Materials

### Oligonucleotides

The oligonucleotides are purchased from Invitrogen without additional purification. The purified oligonucleotide is subjected to electrophoresis in a 15% PAGE-Urea gel, excised from the gel without direct UV irradiation and eluted using the Elutrap electro-separation system (Schleicher and Schuell). The size-purified oligonucleotide are then precipitated with isopropanol, washed with ethanol, dried, and stored at −20° C.

### Methods

The strain of choice is grown in a shaking water bath at 32° C. in LB with 0.4% maltose to mid-exponential phase, (A₆₀₀ is 0.4-0.6). A 30 ml culture is adequate for several recombineering reactions. The culture is harvested by centrifugation and resuspended in 1 ml TM (10 mM Tris base, 10 mM MgSO₄, pH 7.4). The phage to be engineered is added at a multiplicity of infection of 1-3 phages/cell (cell density is assumed to be approximately 10⁸ cells/ml before concentration) and allowed to adsorb at room temperature for 15 minutes (for other phages, it may be desirable to conduct such adsorption at lower temperatures (e.g., at 20° C.-0° C.). Meanwhile, two flasks with 5-ml broth are prewarmed to 32° C. and 42° C. in separate shaking water baths. The infected culture is divided and half-inoculated into each flask; the cultures are incubated an additional 15 min. The 42° C. heat pulse induces prophage functions; the 32° C. uninduced culture is a control. After induction, the flasks are well chilled in an ice water bath and the cells transferred to chilled 35-ml centrifuge tubes and harvested by centrifugation at approximately 6500×g for 7 minutes. The cells are washed once with 30 ml of ice-cold sterile water; the pellet is quickly resuspended in 1-ml ice-cold sterile water and pelleted briefly (30 seconds) in a refrigerated microfuge. The pellet is resuspended in 200-μl cold sterile water and 50-100 μl aliquots are used for electroporation with 100-150 ng PCR product or 10-100 ng oligonucleotide. Electroporation is accomplished using a BioRad E. coli Gene Pulser set at 1.8 mV and 0.1-cm cuvettes. Electroporated cells are diluted into 5 ml 39° C. LB medium and incubated to allow completion of the lytic cycle. The resulting phage lysate is diluted and titered on appropriate bacteria to obtain single plaques. (for more details, see Thomason et al. (2003) “RECOMBINEERING: GENETIC ENGINEERING IN BACTERIA USING HOMOLOGOUS RECOMBINATION,” Curr. Prot. Mol. Biol., pp. 1.16.1-1.16.16.).

### Results of Experiments Using Recombineering

(i) Suppressible mutations are generated by introducing UAG termination codons in essential genes O, P, Q, S, and E. The target phage cII68 acquires these amber mutations at a frequency of 1-3% in a cross with 70-nucleotide-long ss-oligos with the UAG codon at the center. Amber mutants are easily identified as cloudy plaques with a double-layer bacterial lawn (Campbell, A. (1971) GENETIC STRUCTURE, In: Hershey, A. D., Editor, The Bacteriophage Lambda, Cold Spring Harbor Laboratory Press, Cold Spring Harbor, N.Y., pp. 13-44) the lower layer contains the restrictive host W3110 and the top layer contains the infected SupF suppressor host LE392. cII68 lyses both hosts, thereby generating a clear plaque. Amber mutants lyse only the infected LE392 cells and form cloudy plaques because W3110 cells in the lower layer grow to confluence.

(ii) Previous studies of Cro function are based primarily on the use of one missense mutant, cro27. The phage cI^{ts}857 cro27 forms clear plaques at 37° C. but cannot form plaques at either 32 or 42° C. (Eisen et al. (1971) REGULATION OF REPRESSOR SYNTHESIS, In: Hershey, A. D., Editor, The Bacteriophage Lambda, Cold Spring Harbor Laboratory, pp. 239-245). The Cro protein contains three tyrosine residues, and we independently replaced each tyrosine codon with UAG. Screening plaques at 42° C. in a double layer, approximately 2% of total plaques are cloudy. On LE392, the resultant mutants grow at 32, 37, and 42° C., but on W3110 they form plaques only at 37° C.

(iii) An 80-nucleotide oligo is used to generate a 326-bp deletion of the cII gene in c+. This ss-oligo provides 40 bases of homology at each end of the segment to be deleted. λc+normally form turbid plaques. Clear plaque recombinants are found at a frequency of 2%. Sequencing showed that the resulting clear mutant phage carried a deletion exactly corresponding to the original design. This deletion fuses the cII translation initiation codon to the downstream O gene, creating a phage with O at the normal cII location.

(iv) The phage λ rexA and rexB genes are precisely replaced with a bla gene conferring ampicillin resistance. The bla gene is first amplified by PCR using primers with 5′ homology to the flanking regions of the rexAB genes; the PCR product is then targeted to the λ chromosome with recombineering. A phage lysate is grown from the electroporation mix and used to form lysogens. Ampr lysogens are selected and the replacement of the rexAB genes by the bla gene in such lysogens is confirmed by PCR analysis (Yu, D. et al. (2000) (“AN EFFICIENT RECOMBINATION SYSTEM FOR CHROMOSOME ENGINEERING IN Escherichia coli,” Proc. Natl. Acad. Sci. USA 97:5978-5983) and by the ability of the recombinant lysogens to plate T4rII mutant phage (Benzer, S. (1955) “FINE STRUCTURE OF A GENETIC REGION IN BACTERIOPHAGE,” Proc. Natl. Acad. Sci. USA 41:344-354).

(v) Using appropriate PCR primers and the gene SOEing technique (Horton, R. M. et al. (1990) “GENE SPLICING BY OVERLAP EXTENSION: TAILOR-MADE GENES USING THE POLYMERASE CHAIN REACTION,” Biotechniques 8(5):528-535), a linear DNA product is created which contains an intact copy of the wild-type λ P gene adjoining a precise deletion of the entire r en gene but with homology beyond ren in the ninR region of the phage. The construct is targeted to an infecting Pam80 phage; P⁺ recombinants are selected and screened for the ren deletion. P⁺ recombinants are obtained at a frequency of 2%; 20% of these had the deletion.

Analysis of Mutations Arising from the Use of Oligonucleotides in Recombineering.

Recombineering provides an efficient way to manipulate the bacteriophage genome. However, oligo recombination has occasionally been associated with unwanted mutations. To understand the origin and nature of these unwanted mutants, a protocol is designed to score for both true recombinants and unwanted changes. Phage cI^{ts}857 carries a temperature-sensitive mutation in the repressor; thus, this phage forms clear plaques at 37° C. and turbid plaques at 30° C. (Sussman, R. et al. (1962) “SUR LA NATURE DU RÉPRESSEUR ASSURANT L'IMMUNITÉ DES BACTÉRIES LYSOGÉNES,” C.R. Acad. Sci. 254:4214-4216.). Two complementary oligonucleotides 82 residues in length, with wild-type repressor gene sequence, are designed to generate wild-type recombinants in a cross with cI^{ts}857. These oligos cover about 1/10 of the cI coding region and are centered on the cI^{ts}857 allele. The recombinant lysate is diluted and plated on W3110 at either 37° C. or 32° C. At 37° C., c+ recombinant phage form turbid plaques. At 32° C., both parent phage and recombinant phage should form turbid plaques. When plaques from the recombineering cross are grown at 37° C., most are clear, however, 4-13% are turbid as expected of wild-type recombinants. When the recombinant lysate is plated at 32° C., most plaques are turbid as expected, however, a significant proportion, 0.5-2%, are clear. This number is 10-40 times higher than the spontaneous frequency of clear plaques (approximately 0.05%) found in lysates prepared the same way but without the addition of oligonucleotide or with the addition of an oligonucleotide lacking homology.

To understand the source of the unwanted clear mutations, clear and turbid recombinants are purified and the cI gene is sequenced. Fourteen turbid cI+ recombinants isolated at 37° C. have all been corrected for the cI^{ts}857 mutation without additional mutations. However, all clear plaques identified at 32° C. contain other mutations in cI. These mutations are about equally produced by the two oligonucleotides. Twenty-four of twenty-five sequenced have mutations in the region covered by the ss-oligo. Among these 24 mutants, 22 have also converted the cI^{ts}857 allele to wild type. One of these 22 mutants is a G/C to T/A transversion, and the rest are deletions of one or more bases of the cI sequence. The one change outside of the oligo region is a G/C to T/A transversion that retains the cI^{ts}857 allele and that possibly arose spontaneously.

To demonstrate that these mutations are not specific to cI^{ts}857 or to the oligo sequence, the experiment is repeated using wild-type cI+ and complementary ss-oligos from a different region of the cI gene in a cross. These oligonucleotides carry a single silent AT to GC change. As before, clear plaques are found in the lysate following recombineering. The DNA from 16 clear plaques is sequenced, and the sequencing results show that fifteen carry the silent mutation, indicating that they had undergone recombineering. Nine have a single base pair deletion, three have longer deletions, one mutant has an added AT base pair, one shows a C/G to T/A transition, and one has a G/C to A/T base substitution mutation located outside the region covered by the ss-oligo. The one mutant lacking the signature change has a C/G to T/A transition outside the region covered by the ss-oligo and may be a spontaneous clear mutant.

The results presented above suggest that most of the mutations were introduced during synthesis of the ss-oligos. Based on the results and chemistry of synthesis, one would expect that at each position of an oligonucleotide there would be an equal chance of not incorporating the added base (Hecker, K. H. et al. (1998) “ERROR ANALYSIS OF CHEMICALLY SYNTHESIZED POLYNUCLEOTIDES,” Biotechniques. 24(2):256-260; Temsamani, J. et al. (1995) “SEQUENCE IDENTITY OF THE N-1 PRODUCT OF A SYNTHETIC OLIGONUCLEOTIDE,” Nucleic Acids Res. 23(11):1841-1844); Examination of the sequence changes among the frameshift mutations shows that they cluster toward the center of the ss-oligo. The terminal regions lack mutations, suggesting that complete base pairing at the termini may be important for efficient annealing to the phage DNA. To reduce the frequency of frameshift mutations, we further purified the ss-oligos. Purification by HPLC does not reduce the mutation frequency probably because HPLC does not efficiently separate oligos of this length, whereas PAGE-purified oligonucleotides yielded efficient recombineering with fewer frameshift mutations. This result supports the notion that base deletions originating during chemical synthesis of the oligonucleotides are responsible for generating mutations. Single base frameshift deletions occur rarely as spontaneous mutations (Schaaper, R. M. et al. (1991) “SPONTANEOUS MUTATION IN THE Escherichia coli LACI GENE,” Genetics 129(2):317-326). In the above-described examples, deletion mutations formed usually also carried the designed change present on the ss-oligo, suggesting that the frameshift mutations were conferred by the synthetic ss-oligo. Thus, the experimental approach described here provides a simple and sensitive assay for oligonucleotide quality. Recombineering with unpurified synthetic oligonucleotides could also be used to provide an efficient way to introduce random single base deletions at specific sites in genes or regulatory regions. The act of recombineering does not appear to cause random mutagenesis.

When recombineering with the bacterial chromosome, one of two complementary ss-oligos gives more recombinants (Ellis, H. M. et al. (2001) (“HIGH EFFICIENCY MUTAGENESIS, REPAIR, AND ENGINEERING OF CHROMOSOMAL DNA USING SINGLE-STRANDED OLIGONUCLEOTIDES,” Proc. Natl. Acad. Sci. USA 98:6742-6746); Zhang, Y. et al. (2003) “PHAGE ANNEALING PROTEINS PROMOTE OLIGONUCLEOTIDE-DIRECTED MUTAGENESIS IN Escherichia coli AND MOUSE ES CELLS,” BMC Mol. Biol. 4:1-14). This strand bias depends upon the direction of replication through the recombining region with the lagging strand being the more recombinogenic. In the phage crosses, both complementary oligos were equally efficient in promoting recombination at cI. This is likely due to the rolling circle mode of phage DNA replication, which can roll in either direction (Takahashi S. (1975) “THE STARTING POINT AND DIRECTION OF ROLLING-CIRCLE REPLICATIVE INTERMEDIATES OF COLIPHAGE LAMBDA DNA,” Mol. Gen. Genet. 142(2):137-153). Thus, replication forks pass through cI in both directions and neither strand is exclusively leading or lagging.

In the cross with lambda cI^{ts}857, mottled plaques at 37° C. are observed, indicating that the lambda DNA was packaged with a heteroduplex allele in cI (Huisman, O. et al. (1986) “A GENETIC ANALYSIS OF PRIMARY PRODUCTS OF BACTERIOPHAGE LAMBDA RECOMBINATION,” Genetics 112(3):409-420). Six independent mottled plaques were purified and found to give rise to a mixture of turbid and clear plaques. Sequence analysis shows that in all cases the turbid plaques had incorporated the wild-type allele, whereas the clear plaques retained the original cI^{ts}857 mutation, indicating that the oligonucleotide paired with the phage chromosome and was incorporated without mismatch correction. These heterozygous phages are generated in recA mutant crosses, which suggests that the ss-oligo is annealed by Beta protein to single-strand gaps at the replication fork (Court, D. L. et al. (2002) “GENETIC ENGINEERING USING HOMOLOGOUS RECOMBINATION,” Annu Rev Genet. 36:361-388; Stahl, M. M. et al. (1997) “ANNEALING VS. INVASION IN PHAGE LAMBDA RECOMBINATION,” Genetics. 147(3):961-977).

### Example 3

### Use of Bacteriophage λ-Based ex-vivo Genetic System to Identify and Study Protein-Protein Association

### Materials

ADL1 (cI^{ts} Dam); λDam imm21 nin5 (Sternberg and Hoess, 1995, Proc. Natl. Acad. Sci. USA 92:1609-1613) was used for constructing DL1, from which the λ-A2 (cI^{ts} Dam kan^{r}) and λ-A3 (cI^{ts} Dam cml^{r}) vectors were made. DY330, is W3110 ΔlacU169 gal490 pglΔ8 cI857Δ(cro-bioA). LE392 (supE,F⁺), W3110 (sup⁻), BM25.8 (supE, Cre/Lox⁺), pDC3 (amos/paper/Dis110AA). The proteins wtCUE (50 amino acids), Ubiquitin (30 amino acids), CUEM419D (non-binding mutant), acidic and basic aptamers (New England Peptide Inc) were obtained from the US NIH and used for titration of fusion display phage binding.

### Methods

### Construction of Display Phages

A schematic of the genetic steps used in the construction of the display phage is shown in FIG. 3. The details of the genetics behind this process are covered in Gupta, A. et al. (2003) (HIGH-DENSITY FUNCTIONAL DISPLAY OF PROTEINS ON BACTERIOPHAGE LAMBDA,” J. Mol. Biol. 334(2):241-254), Thomason et al. (2003) (“RECOMBINEERING: GENETIC ENGINEERING IN BACTERIA USING HOMOLOGOUS RECOMBINATION,” Curr. Prot. Mol. Biol., pp. 1.16.1-1.16.16) and Court, D. L. et al. (2002) “GENETIC ENGINEERING USING HOMOLOGOUS RECOMBINATION,” Annu. Rev. Genet. 36:361-388.

Overnight cultures of E. coli BM25.8/pDC3-X were diluted to OD₆₀₀ 0.075 in LB supplemented with 50 μg/ml ampicillin (Amp) and 12.5 μg/ml chloramphenicol (Cml) to mid-log phase (OD₆₀₀ 0.3=1×10⁸ cells/ml). The cells were collected at 4400×g for 7 min then resuspended in 1 ml of diluted A2_{K} or A3_{C} vector phage in TMG (Tris.HCl, MgSO₄.7H₂O and gelatin; KD Medical, Columbia, Md.) at a multiplicity of infection of 1. Infection, recombineering and gene expression is allowed to proceed for 1 h at room temperature (RT), then the cells are diluted in 1 ml Luria Broth (LB) supplemented with 50 μg/ml Amp and 12.5 μg/ml Cml or 30 μg/ml Kan, adjusted for the final 2 ml volume. The lysogens are cultured at 32° C. for 3-4 h, induced to lyse by shifting the temperature to 42° C. and the cleared lysate is treated with 10% chloroform. One hundred and fifty microliters of this lysate is used to infect 5 ml of fresh overnight recovered log-phase E. coli LE392 supE,F⁺ (or E. coli W3110 sup⁻) in [LB+0.4% maltose+10 mM CaCl₂] at RT. After 1 h, the cells are spread on agar supplemented with either Cml/Amp or Kan/Amp, and the plates are incubated overnight at 32° C. A single colony is isolated and cultured in 1 ml of LB supplemented with 50 μg/ml Ampicillin and 12.5 μg/ml Cml or 30 μg/ml Kan (depending on the antibiotic resistance determinant of the background vector phage) for 5.5 h at 32° C. The cells are induced to lyse by shifting to 42° C., and the cleared lysate is filtered through 0.22 μm Millipore membranes to remove bacterial debris.

The Display Phages are assayed for recombination first by their ability to form plaques on the non-suppressor strain E. coli because the vector Dam mutants used for recombineering can not infect this host. A Spot Test (Sambrook et al., Molecular Cloning: A Laboratory Manual, Cold Spring Harbor Press, 1989) is performed, with plaque formation on E. coli W3110 (sup⁻) used to verify that Cre/Lox-directed recombination had occurred between the parent phage and the wild type (wt) D gene contained in the pDC3 plasmid. A single plaque represents a single lambda that has undergone initial infection followed by cycles of lysis and re-infection of surrounding cells. The vector Dam phages obtained from the E. coli LE392 (supE,F⁺) lysate will also have a non-display D protein on their heads due to suppression that allows for the initial infection event, however subsequent rounds of phage growth in the sup⁻ strain will not occur to form the plaque if the wt D from a D-fusion is not present.

E. coli LE392 supE⁺,F⁺ is used for initial phage growth because its suppressor functions allow for mixed expression of both the wtD (from the parental Dam gene) and fusion D-display protein (from recombination with pDC3), which may be necessary to avoid phage instability. It is believed that suppression of the Dam mutation by the E. coli LE392 supF function, that replaces the amber STOP with a tyrosine residue, produces a D protein that is deleterious to phage stability. Since the D protein can be supplied in trans (Zanghi, C. N. (2005) “A SIMPLE METHOD FOR DISPLAYING RECALCITRANT PROTEINS ON THE SURFACE OF BACTERIOPHAGE LAMBDA,” Nucleic Acids Research 33(18):160), a preponderance of tyrosine suppressed D protein can result in a lysate-wide killing of lambda (Table 1). To detect display of the gene product, the Display Phages are examined for D-fusion expression and assayed for protein activity, where applicable.

TABLE 1

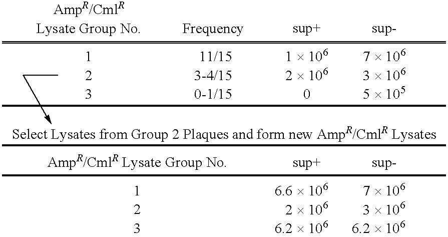

Table 1 shows sample results for λD-Acid_{c} construction and the decontamination of lysogens through plaque purification. Non-recombineered contaminating vector phages can be selected out by their differential plaquing behavior on suppressor⁺ and suppressor⁻ strains. The higher plaque counts on E. coli LE392 supE, F⁺ is due to the presence of vector phages in the lysate. Plaque purification removes this background. Plaque purified lysate 3 from Amp^{R}/Cml^{R} Lysate Group 2 was chosen to make a working stock. There are three groups of phage lysates that were typically found following the initial infection of E. coli LE392 with the E. coli BM25.8 lysate: (1) those with high vector contamination, (2) those that are near pure for recombineered phage, and (3) those that contain potentially tyrosine substituted phages. Those phage lysates that fall into Group2 s are then plaque purified by standard methods, filtered and titred again on E. coli W3110 and E. coli LE392. Phages must produce equivalent numbers of plaques on both E. coli strains in order to be considered pure of background vector phages. To detect display of the gene product, the Display Phages are examined for D-fusion expression and assayed for protein activity, where applicable.

Protein Association Assay

Display phages are assayed for their ability to associate with other phages bearing a potential binding partner, with a non-binding partner and with a non-display D vector phage (i.e. λA2 and λA3). The production of double antibiotic resistant (Cml^{r}/Kan^{r}) multilysogen E. coli LE392 is used to mark positive fusion D display protein-protein interaction. First, the phages are combined to yield a chosen MOI (a typical initial range is 0.002-0.04), and allowed to associate for 5 minutes at room temperature (RT) prior to dilution with 220 uL of Salted Association Buffer (SAB; 20 mM Tris.HCL_{pH}7.4, 10 mM CaCl₂, 10 mM MgCl₂ and 100 mM NaCl). After 10 minutes at room temperature, 1×10⁸ of fresh log-phase E. coli LE392 recovered in [LB+0.4% maltose+10 mM CaCl₂] is added. Phage-phage association, co-infection and antibiotic gene expression are allowed to proceed for 45 min-1 h at RT, followed by plating on agar supplemented with 10^{ug}/_{mL} Cml+30^{ug}/_{mL} Kan. Plates are incubated overnight at 32° C. for lysogen formation.

The Display Phages are also assayed with vector phage (i.e., λD-Base_{C} with λA2K), with themselves (i.e., λD-Base_{C}+λD-Base_{K} in cases where homodimerization does not occur) as well as a non-binding partner phage (i.e., λD-Base+λD-Ubiquitin) to eliminate the prospect of non-specific interactions.

### Results and Discussion

### Construction of a Phage-Based System for Studying Protein-Protein Interactions

To develop a general strategy for assaying interactions of proteins displayed on the lambda virion, the well studied CUE:Ubiquitin protein pair and an uncharacterized Acid:Base [Gly-Glu]₄:[Gly-Arg]₄ [SEQ ID NO:4 and SEQ ID NO:5, respectively] aptamer pair were employed. Ubiqitinization of proteins plays a major signaling role during such cellular processes as cycling, stress response, DNA repair, transcription and gene silencing. Proteins (i.e. those targeted for degradation within the cell) carrying a ubiquitin modification are recognized by proteins containing UEV, UBA, UIM and CUE domains, the latter of which is involved in recruiting proteins for ER degradation. The CUE domain (50 amino acid residues), as characterized by the conserved MFP and LL motifs, binds to monoubiquitin (30 amino acid residues) as a monomer at ubiquitin's hydrophobic core with high affinity of K_{D}=20 μM. However, CUE can also bind Ubiquitin as a dimer with an apparent K_{D}=1.2 μM. Both of these binding constants are stronger than the theoretical lower affinity limit of 50 μM determined by biopanning assays for detection of protein association (Zucconi, A. et al. (2001) “SELECTION OF LIGANDS BY PANNING OF DOMAIN LIBRARIES DISPLAYED ON PHAGE LAMBDA REVEALS NEW POTENTIAL PARTNERS OF SYNAPTOJANIN,” J. Mol. Biol. 2001 307(5):1329-1339). Since both proteins are small (i.e. their genes are within the 2 Kb optimal limit for use with the A2 and A3 vectors (Hoess, R. H. (2001) “PROTEIN DESIGN AND PHAGE DISPLAY,” Chem Rev 101:3208-3218; Hoess R H. (2002) “BACTERIOPHAGE LAMBDA AS A VEHICLE FOR PEPTIDE AND PROTEIN DISPLAY,” Curr. Pharm. Biotechnol. 3(1):23-28) and the they bind with an affinity above the minimal threshold, the CUE:Ubiquitin protein pair is a suitable model to use for the development and validation of our lambda 2-Hybrid system. Additionally, CUE can bind itself via an alpha helix interface with a K_{d} (dimerization) of 1 mM, which could serve in the role of a lower affinity standard (Prag, G. et al. (2003) “MECHANISM OF UBIQUITIN RECOGNITION BY THE CUE DOMAIN OF VPS9P,” Cell 113(5):609-620).

Successful LoxP/Cre-directed recombineering between the wt Lox and mutant Lox sites of the vector phages (λA2 or λA3) with those of the plasmid results in Amp^{r}, D-display phages. The pDC3 plasmid used for D-display contains an MCS downstream of the first 110 amino acid residues of the D protein that allows for fusion of a target protein through a three amino acid linker at the C-terminal end of D. Since during lambda maturation the D protein attaches to the E protein on the outer surface of the virion head after head formation, the character of the fusion protein is not deterred during assembly nor does the fusion protein interfere with formation of the head. The wild type (wt) D used for fusion serves as a selectable marker for successful recombineering of large pieces of DNA (10% of wild type λ DNA) since λ can be viable in the absence of gpD only if their 48.5 Kb genome (NCBI GI accession # 9626243) is shorter in length (by approximately 2 Kb), and in this system the phage genome is maintained at or above full length throughout the process. The Dam mutation that remains in the display phage genome serves to both decrease extreme expression of large (1000⁺ amino acid residues) polypeptides that could impose excessive weight on the display phage head as well as ensures the phage head is not destabilized by the added pressure of an enlarged genome due to a longer gene insert (Maruyama, I. N. et al. (1994) “LAMBDA FOO: A LAMBDA PHAGE VECTOR FOR THE EXPRESSION OF FOREIGN PROTEINS,” Proc Natl Acad Sci USA. 91(17):8273-8277; Terry, T. D. et al. (1997) “ACCESSIBILITY OF PEPTIDES DISPLAYED ON FILAMENTOUS BACTERIOPHAGE VIRIONS: SUSCEPTIBILITY TO PROTEINASES,” Biol. Chem. 378(6):523-530; Mikawa, Y. G. et al. (1996) “Surface display of proteins on bacteriophage lambda heads,” J. Mol. Biol. 262(1):21-30). The PGGSG (SEQ ID NO:1) amino acid linker between D and the fusion display protein allows for a higher degree of movement of the peptide for it to associate with other proteins and function while fused to the virion head.

The recombineering of each display peptide into the vector λA2 or λA3 genome was successfully accomplished, as demonstrated by the production of Amp^{r}/Cml^{r} or Amp^{r}/Kan^{r} E. coli LE392 lysogens, and the ability of the phages to form a plaque on a non-suppressor host strain (i.e. E. coli W3110 that does not allow the parental Dam mutant to form a plaque). The display phages possess unusually shaped prolate heads, likely due to interactions between the displayed peptides or the added weight suffered by the phage head. In the unique case of Ubiquitin, the phage head is surrounded by uncharacterized vesicles released from the lysed E. coli host cells. The ubiquitin protein has been demonstrated to bind miscelles from lysed yeast cells. The presence of these vesicles bound to only the λD-Ubiquitin display phage aids in the validation of the presence of peptide, and may also be interpreted to be the first evidence demonstrating that the protein is in fact able to retain its natural function as a fusion D-display protein.

### Protein Association Assays

Although most prophage genes are repressed in the lysogenic host, independently controlled gene functions that are carried by λ can be expressed (Oppenheim, A. B. et al. (2005) “SWITCHES IN BACTERIOPHAGE LAMBDA DEVELOPMENT,” Annu Rev Genet. (Epub ahead of print); Kobiler, O. et al. (2005) “QUANTITATIVE KINETIC ANALYSIS OF THE BACTERIOPHAGE LAMBDA GENETIC NETWORK,” Proc. Natl. Acad Sci USA. 102(12):4470-4475; Thomason et al. (2003) (“RECOMBINEERING: GENETIC ENGINEERING IN BACTERIA USING HOMOLOGOUS RECOMBINATION,” Curr. Prot. Mol. Biol., pp. 1.16.1-1.16.16); Court, D. L. et al. (2002) “GENETIC ENGINEERING USING HOMOLOGOUS RECOMBINATION,” Annu. Rev. Genet. 36:361-388). The analysis was restricted to genes that confer antibiotic resistance (kanamycin or chloramphenicol carried in the vector λA2 and λA3 phages, respectively) to the host bacteria. These resistance genes were introduced at identical sites between the genes R and cos within the phage genome to eliminate the possibility of obtaining λ phage recombinant carrying both resistance genes. In order to simplify the rescue of the prophage for further studies, a temperature-sensitive repressor was employed (cI^{ts}857; Gupta, S. et al. (2001) “MAPPING OF HIV-1 GAG EPITOPES RECOGNIZED BY POLYCLONAL ANTIBODIES USING GENE-FRAGMENT PHAGE DISPLAY SYSTEM,” Prep Biochem Biotechnol. 31(2):185-200; Gupta et al. (2003)(“HIGH-DENSITY FUNCTIONAL DISPLAY OF PROTEINS ON BACTERIOPHAGE LAMBDA,” J. Mol. Biol. 334(2):241-254), which allows lytic development upon shifting a lysogenic culture to an elevated temperature (32° C. to 42° C.).

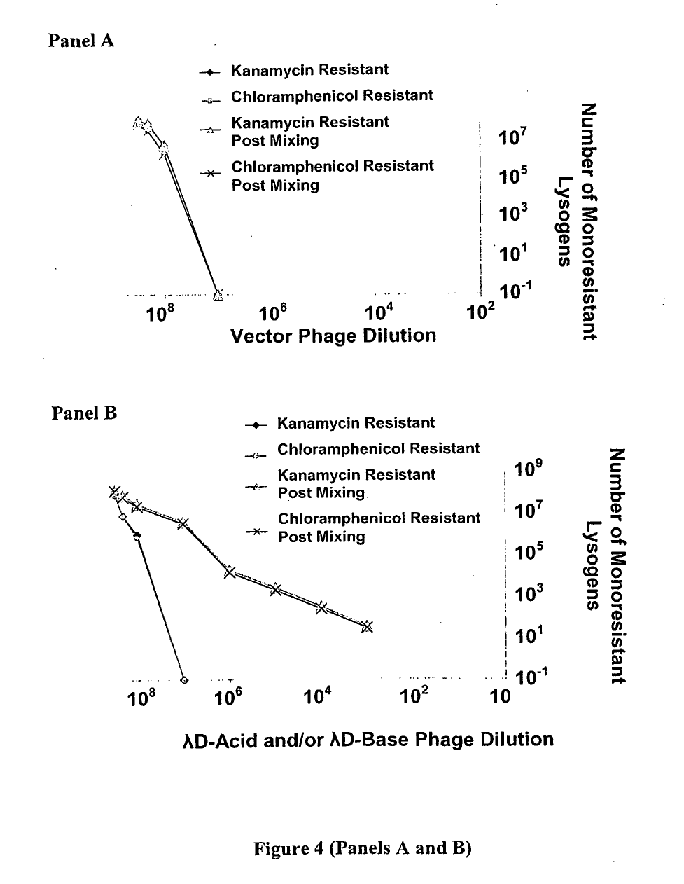

The display phages were first assessed for viability (FIG. 4). The ability of all the display phages to productively infect cells is comparable to that of the non-display vector phages λA2 and λA3 (FIG. 4; Panel A). When used independently to infect host cells, λD-Acid and λD-Base were able to produce monolysogens, demonstrating each of these phages is still viable even though the D protein is fused to a highly charged aptamer (FIG. 4; Panel B). λD-CUE and λD-Ubiquitin, which carry comparatively larger 50 amino acid and 80 amino acid D-fusions, respectively, are also viable (FIG. 4; Panel C). However, a striking difference in transduction efficiency, and the first indication of phage-phage association, is observed when the Display phages are incubated with a binding partner phage prior to selection with a single antibiotic. Since infection of a host cell by λ at an MOI of 1 or less leads to 99% lytic growth but at an MOI of 2⁺ makes mostly stable lysogens, the large increase in mono antibiotic-resistant lysogens and the marked decrease in the input phage necessary to form lysogens post-mixing is likely due to display phage association and subsequent dual infection (FIG. 4; Panel D). With only one antibiotic resistance being assayed, the colony count is reflective of cells that are infected by (i) a single phage (i.e. monolysogen), (ii) two different phages (i.e. double resistant multilysogen), or (iii) two similar phages (i.e. monoresistant multilysogen) due to aggregates formed by the associating phages.

Investigations were then conducted to determine whether the display phages were able to associate with a binding partner in a highly productive manner; that is, able to produce a bacterial cell expressing both the Cml^{r} and Kan^{r} resistance markers and at what efficiency when the phage input is far below the cellular input. Stable lysogeny of, and especially dual infection by, vector phages is rare when cells far outnumber phage due to low levels of lambda cII protein (that is essential for lysogeny) that cannot overcome the high bacterial protease levels present in the log phase cells (Oppenheim, A. B. et al. (2005) “SWITCHES IN BACTERIOPHAGE LAMBDA DEVELOPMENT,” Annu Rev Genet. (Epub ahead of print); Kobiler, O. et al. (2005) “QUANTITATIVE KINETIC ANALYSIS OF THE BACTERIOPHAGE LAMBDA GENETIC NETWORK,” Proc. Natl. Acad Sci USA. 102(12):4470-4475). The lack of interactions between the non-reactive fusion display or wtD proteins practically eliminates simultaneous infection, which is essential for lysogeny.

The results of phage-phage association studies are illustrated in FIG. 4, Panel E, and selective studies are summarized in Table 2. As anticipated, there was no interaction between the two vector phages (λA2 and λA3) at an MOI of 0.1 (1×10⁷ phages infecting 1×10⁸ cells). At an MOI of 1 (or 1×10⁸ phages), when the phages far outnumber the cells, the vector phages were able to form only 600 Cml^{r}/Kan^{r} double resistant lysogens at a very low level of frequency (600/˜10⁸) (Table 2, Exp#3). In stark contrast to the vector phages, the double resistant multilysogens begin to contribute to the stable lysogen population at an MOI of 0.0001 for λD-Acid:λD-Base and 0.0001 for λD-CUE:λD-Ubiquitin. At an MOI of 0.00025 (or 2.5×10⁴ phages infecting 1×10⁸ cells), λD-Acid:λD-Base forms 150 Cml^{r}/Kan^{r} double resistant lysogens. At an MOI of 1 (1×10⁸ phages), this number rises to approximately 2.4×10⁵ double resistant lysogens (Table 2, Exp#6). For the λD-CUE:λD-Ubiquitin binding pair, approximately 150 Cml^{r}/Kan^{r} double resistant lysogens are formed at an MOI of 0.008 (or 8×10⁵ phages infecting 1×10⁸ cells). At an MOI of 1 (or 1×10⁸ phages) the number of lysogens rises to 1.5×10⁵ (Table 2, Exp#9). Though still quite sensitive, the slightly higher phage input required of the λD-CUE/λD-Ubiqitin partners to effect similar levels of Cml^{r}/Kan^{r} cell formation as λD-Acid/λD-Base can be due to (i) a lower number of these larger polypeptides able to be expressed on the virion head, (ii) inhibitory physical constraints on this protein pair that may not be a factor in the binding between the smaller aptamers or (iii) a lower affinity between CUE:Ubiquitin proteins than that between the charged aptamers. It has been shown that the number of fusion D-display proteins on the virion head decreases ( 350/405, 210/405, 154/405) as the protein size increases (22, 31, 39 KDa), though display of the proteins is still highly multivalent (Gupta et al. (2003)(“HIGH-DENSITY FUNCTIONAL DISPLAY OF PROTEINS ON BACTERIOPHAGE LAMBDA,” J. Mol. Biol. 334(2):241-254). Table 2 provides a summary of the effects of preincubation on the number of monoresistant lysogens and the striking disparity in double resistant multilysogen formation between vector and display phages (Cml^{r}=chloramphenicol resistant; Kan^{r}=kanamycin resistant; MOI=multiplicity of infection; NA=not applicable).

TABLE 2

| | | | Number of | Number of | Number of |
|---|---|---|---|---|---|
| | | | Cml^{r} | Kan^{r} | Cml^{r}/Kan^{r} |
| Expt. | Input Phage | MOI | Cells | Cells | Cells |
| 1 | Vector 2 | 0.1 | NA | 0 | 0 |
| 2 | Vector 3 | 0.1 | 0 | NA | 0 |
| 3 | Vector 2 + | 2 (0.1) | 0 | | 0 |
| | Vector 3 | 2 (1) | 0 | | 6 × 10² |
| 4 | Acid | 0.1 | NA | 3 × 10⁶ | 0 |
| 5 | Base | 0.1 | 0 | 3 × 10⁶ | 0 |
| 6 | Acid + Base | 2 (0.1) | 3 × 10⁶ | | 1.1 × 10⁴ |
| | | 2 (1) | 2 × 10⁷ | | 2.4 × 10⁵ |
| 7 | CUE | 0.1 | 0 | 3 × 10⁶ | 0 |
| 8 | Ubiquitin | 0.1 | NA | 3 × 10⁶ | 0 |

| | | | | |
|---|---|---|---|---|
| 9 | CUE + Ubiquitin | 2 (0.1) | 2.5 × 10⁶ | 2 × 10³ |
| | | 2 (1) | 2 × 10⁷ | 1.5 × 10⁵ |

A small effect was observed on double resistant lysogen formation by aggregation and dilution. The titer was observed to decline by 2-fold following incubation and centrifugation of the partner pairs, and dilution of these aggregates appeared to aid in productive protein association. The optimal percentage of phage available to undergo productive interactions was found at the point where the number of input display phages was approximately 2×10³ pfu/mL with cells at a 5×10⁵ excess (i.e. 10³ of each display phage partner infecting 10⁹ cells in a 0.5 mL reaction). This effect of dilution is possibly due to a decrease in non-productive clumping of display phages. It should be noted that in these studies all phages are members of a single binding pair. Aggregation and its effect may not be a factor when challenging a bait phage with more than one prey, such as in panning a single display phage against a heterogenous group or a library of fusion display peptides. Rather, such tight association due to multivalent expression of fusion display proteins may prove to be a beneficial characteristic of the λ-2Hybrid system when searching for specific associations in the diluted environment of a pool or library of unknowns.

The ability of all binding partners to ‘find’ each other was found to be insensitive to presence of excess non-specific background phages and the strength of fusion display protein association to be directly proportional to the degree of Cml^{r}/Kan^{r} cell formation. There was no interaction observed between the display phages with vector (non-display) phages, and double resistant lysogen formation was not deterred by the presence vector phage at a ratio of 1(display phage pair):200(non-display phage). Additionally, the presence of BSA up to 50 mg/mL did not impact protein-protein association observed. At lower MOI's, the display phages are shown to produce Cml^{r}/Kan^{r} double resistant multilysogens when associated specifically with (and for the majority only with) their binding partner.

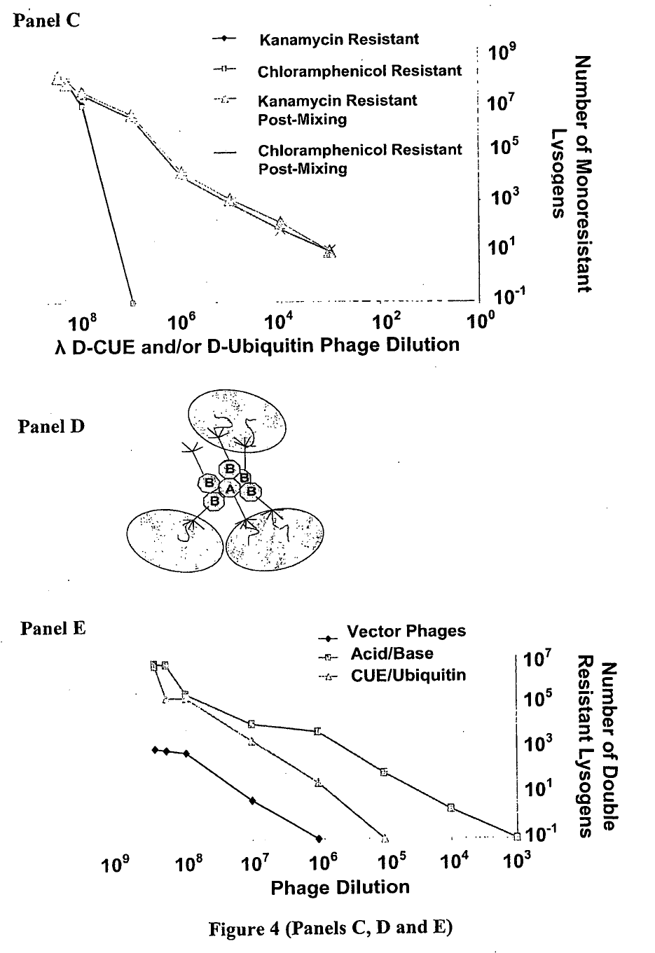

Once the threshold for λ-λ interactions were determined and an infection protocols were established for each binding partner pair, we asked how the system responds to increasing MOI's (also referred to a decreasing dilution). The findings, shown in FIG. 5 revealed that the differences in protein binding strength could be readily distinguished as phage input increases. For binding partners (λD-Acid:λD-Base and λD-CUE:λD-Ubiquitin) productive co-infection begins at an MOI well below 1 and the number of Cml^{r}/Kan^{r} lysogens rises sharply as display phage inputs increase. This is not the case for assisted, chemical or incidental interactions where the number of double resistant lysogens is significantly lower (λD-Ubiquitin:λD-Ubiquitin), or essentially zero (λD-CUE:λD-Acid and λA2:λA3) at an MOI below 1, and the Cml^{r}/Kan^{r} colony count fails to rise as sharply as the number of input phages is increased.

At excessive phage input (MOI of 3-5), the levels of Cml^{r}/Kan^{r} was found to remain approximately constant for fusion display phage that undergo specifical protein-protein association. This plateau may be the result of (i) aggregation of the binding partners as the phage input increases while the reaction volume remains constant, (ii) bias towards monoresistant multilysogen formation at high phage input levels, (iii) be reflective of a common percentage of the input phage that are never available for binding due to clumping, (iv) to steric hinderances that do not allow a protein-associated phage partner to orient its tail on the cell surface, or (v) steady decrease in the availability of one of the partners that is the limiting factor in a given pair. These characteristics could potentially be used in combination to assess how strong a protein-protein interaction is by assessing how few of the display phages are required to begin forming double resistant multilysogens (i.e. how far below an MOI of 1) and examining the rise in Cml^{r}/Kan^{r} formation as the phage inputs are increased (i.e. a sharp rise versus an insignificant or very shallow rise). The greater the affinity of binding between the two proteins, the less input phage needed. Protein-protein interactions that meet both criteria for strong binding can then be assessed as likely “true” positives that associate directly and specifically.

Very little cross-association is seen between Display Phage pairs, with the exception of an assisted interaction between the Ubiquitin fusion display phage and a weak, potentially chemical, association between λD-CUE:λD-Acid. The unusually high level of homodimerization observed for λD-Ubiquitin, which is known occur at lower levels than for gpCUE (Gali), is likely due to “bridging” of the two ubiquitin molecules by those uncharacterized vesicles seen bound to λD-Ubiquitin in the EM (see Construction of Display Phages). At higher MOI's, the number of Cml^{r}/Kan^{r} formed from this aided association levels off at a value far below that of λD-CUE:λD-Ubiquitin. The instability of the interactions with this form of a ‘bridging molecule’ is possibly responsible for this phenomenon or is reflective of a high K_{D} for the weak interaction. The binding between λD-CUE and λD-Acid is likely incidental, and their non-specific interaction is reflected in failure of the number of Cml^{r}/Kan^{r} lysogens formed from this pair to increase at higher MOIs.

### Specific Titration of Phage-Phage Interactions by Competing Polypeptides

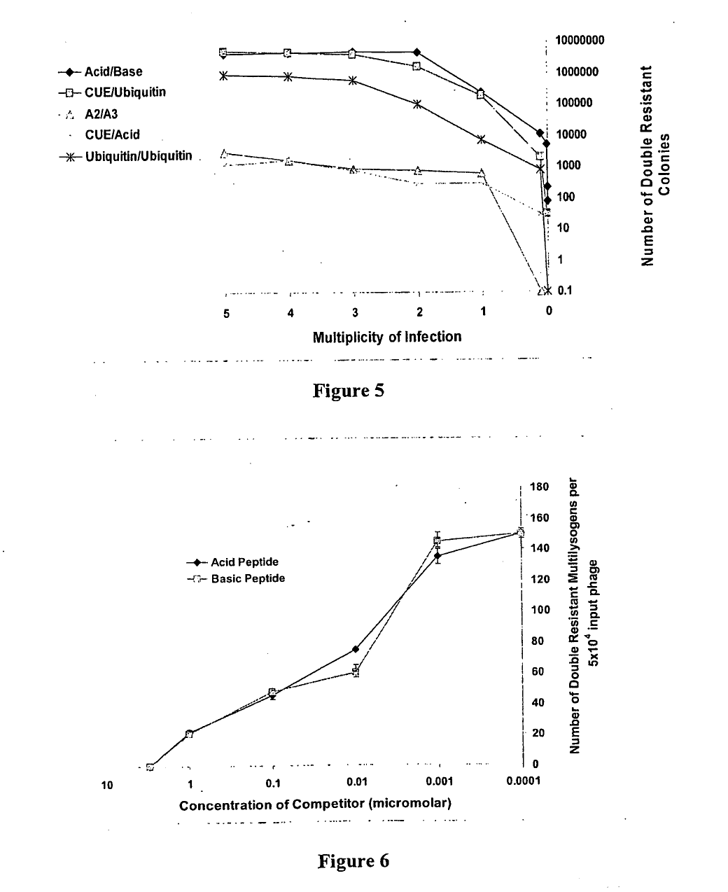

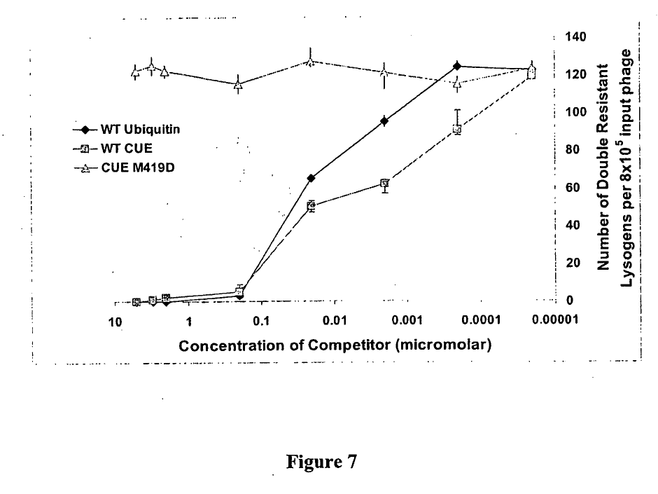

To further verify that the observed binding reactions are specific, investigations were conducted as to whether the binding partners could be specifically titrated with free peptides corresponding to the fusion display proteins. Synthetic acidic and basic polypeptides matching those displayed on λD-Acid and λD-Base were used to compete with the aptamer interaction. An apparent K_{D} of 10 nM was obtained with both aptemers, as calculated as the concentration of free peptide that results in an IC₅₀ (FIG. 6). Arginine and Glutamic acid were also found to inhibit this interation at 4.6 mM and 0.679 mM, respectively. The λD-Cue:λD-Ubiquitin pair was shown to be competed by free gpUbiquitin with an apparent K_{D} of 20 nM and free wild type gpCue at 2 nM. The K_{D} of free gpCue:gpUbiquitin has been calculated in vitro to be 1.2 μM for dimericCue:Ubiquitin and 1.1 mM for monomericCue:Ubiquitin. In contrast, the non-binding mutant gpCueM419D was unable to challenge the λD-CUE:λD-Ubiquitin interaction to any degree, even at a concentration of 1 mM (FIG. 7). The lack of inhibition by mutant gpCueM419D validates both the specificity of the interaction the “native” biochemical nature of the fusion display proteins of λD-Cue:λD-Ubiquitin since the physical presence of a non-binding gpCUEM419D is not able to act as a competitor. There was no cross-inhibition found of λD-Acid:λD-Base with wild type gpUbiquitin, gpCUE or gpCUEM419D, nor is λD-CUE:λD-Ubiquitin titrated by the acidic or basic aptamers.

### A Novel Catastrophic Phage-Phage Interaction

Investigations were conducted to determine the role played by incubation time in the yield of double resistant lysogens. To assay if extending the time of the interactions between the display phages increases the number of Cml^{r}/Kan^{r} colonies, the Display Phage lysates were incubated either separately or as a mixture at high concentration for 30-60 min at either 4° C. or room temperature. It was found that whereas the individual lysates are stable, incubating the display phage partners for an extended time (>1.25 h) prior to cell infection leads to a rapid loss in the number of Cml^{r}/Kan^{r} lysogens (data not shown). However, this loss of transduction efficiency was not found with the vector (non-display) phages suggesting that the interacting protein domains are responsible for phage inactivation. Indeed, electron microscopy scans of such display phage mixtures incubated for >1.25 h revealed the formation of aggregates of broken down phage particles and spherical head particles reminiscent of proheads. It is possible that the affinity between the interacting D fusion protein pairs is higher than that of the major capsid protein D with the minor capsid protein E (Yang et. al. 2000). It is suggested that these interacting forces between the engineered phages, which may be initiated by “undressing” the D-fusion proteins from the capsids, lead to their destruction and visualized aggregation of the bursting phage heads. Indeed, as described above, the addition of excess of pure interacting domains competes with phage-phage interaction and stabilizes both phages. Evolutionary forces most probably selected for capsid proteins that do not possess exposed regions for unwanted strong interactions. Further, that modified versions of these macromolecular interactions may have evolved to promote eukaryotic virus disassembly upon entry into host cells.

### CONCLUSION

The successful display of active polypeptides fused to the lambda D protein has been shown previously (Maruyama, I. N. et al. (1994) “LAMBDA FOO: A LAMBDA PHAGE VECTOR FOR THE EXPRESSION OF FOREIGN PROTEINS,” Proc. Natl. Acad. Sci. USA 91(17):8273-8277, Sternberg, N. et al. (1995) “DISPLAY OF PEPTIDES AND PROTEINS ON THE SURFACE OF BACTERIOPHAGE LAMBDA,” Proc. Natl. Acad. Sci. USA 92(5):1609-1613; Mikawa, Y. G. et al. (1996) “SURFACE DISPLAY OF PROTEINS ON BACTERIOPHAGE LAMBDA HEADS,” J. Mol. Biol. 262(1):21-30). Moreover, an increased solubility of insoluble proteins through phage display (i.e. scFv) has been reported in addition to an increased bias for soluble versions of randomized antibodies. Recently, lambda was found to be more advantageous than M13 in construction of a complex hepatitis C virus cDNA library used for natural ligand discovery (Santini, C. et al. (1998) “EFFICIENT DISPLAY OF AN HCV CDNA EXPRESSION LIBRARY AS C-TERMINAL FUSION TO THE CAPSID PROTEIN D OF BACTERIOPHAGE LAMBDA,” J. Mol. Biol. 282(1):125-35). The Display System discussed here represents a novel and powerful strategy for assaying the interaction of proteins that is sensitive, specific and able to be titrated. The present invention demonstrates that lambda display is also compatible with a 2-Hybrid approach for elucidating protein-protein interactions. The Examples demonstrate the ability to display three very different peptides without destroying the phages' viability or the peptides' natural function. Moreover, we present a novel selection system based upon simple antibiotic resistance, and an ex-vivo platform that holds many advantages over other cellular, phage and immobilization systems. The specificity of the system has been shown through the low frequency of non-partner Cml^{r}/Kan^{r} lysogens obtained (i.e. λD-Cue and λD-Acid), the extremely low level of non-specific background, the lack of association with non-display vector phages and the lack of competition by non-specific free peptides.

The lambdoid phage-based 2-Hybrid platform of the present invention may be used successfully for library screening, binding affinity optimization (especially for scFv studies), mutation-based antibody affinity maturation based on simple dilution and free from need for expensive rabbit-based antibody production, and drug discovery (both agonistic and antagonistic). Protein-protein interactions comprise a vast group of targets for therapeutic intervention. The present invention offers the validation of a viable alternative for studying protein interactions that is useful for carrying out a wide range of selection assays with proteins that cannot be studied within the context of the Yeast cell, that are too large for M13 or T7, that can not be secreted (M13) or that are not compatible with silica-fixing. Additionally, the lambdoid phage-based 2-Hybrid platform of the present invention provides a simple way for independent verification of protein-protein interaction determination. In contrast to the yeast two-hybrid system, the present invention is applicable in almost any molecular lab, is carried out ex-vivo free of high concentrations of cellular protein components and the protein-protein interactions are scored independently of specific gene expression. Unlike the M13 and T7 systems, lambda is free of size constraints and membrane considerations (a particularly important consideration in antibody studies). In contrast to previous lambda display studies using protein immobilization-based panning, this approach does not require extensive and elaborate protein immobilization prior to studies, or harsh chemical treatments that can prevent or disrupt protein binding and destroy target peptides. Since detection of a positive interaction can be obtained at low MOI's, this lambda based detection has a natural propensity towards the detection of a low affinity interaction and poorly represented peptides (1:200 in the population is detectable). Being ex-vivo, limitations such at poor folding in an E. coli cellular environment (i.e. disulfide bonds that require a reducing environment or proteins that require a chaperone) one can supply all necessary chemical species in solution and proteins via expression vectors. Such options are not realized with any other protein-protein association platform in production today. For gene transfer applications through phage internalization (Di Giovine, M. et al. (2001) “BINDING PROPERTIES, CELL DELIVERY, AND GENE TRANSFER OF ADENOVIRAL PENTON BASE DISPLAYING BACTERIOPHAGE,” Virology 282(1):102-112; Larocca, D. et al. (1999) “GENE TRANSFER TO MAMMALIAN CELLS USING GENETICALLY TARGETED FILAMENTOUS BACTERIOPHAGE,” FASEB J. 13:727-734), the lambda phage is also more advantageous than the M13 in that it is similar in shape and size to mammalian viruses and has a large dsDNA genome in contrast to the smaller and ssDNA of M13 (Hoess R H. (2002) “BACTERIOPHAGE LAMBDA AS A VEHICLE FOR PEPTIDE AND PROTEIN DISPLAY,” Curr. Pharm. Biotechnol. 3(1):23-28). The novel lambdoid phage 2 Hybrid system of the present invention may be used to study protein-DNA binding, gene regulation, the kinetics of binding, drug-based inhibition of protein signaling, biological processes requiring macromolecular recognition and to deciphering the multitude of binding partners within a regulatory protein complex.

All publications and patents mentioned in this specification are herein incorporated by reference to the same extent as if each individual publication or patent application is specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of identifying or assessing a binding interaction between a target molecule and a target-binder molecule comprising the steps of:
(a) incubating host cells under conditions permissive for lambdoid phage infection of said host cells with
(1) a first lambdoid phage preparation, said preparation comprising first lambdoid phages that display a target molecule, and
(2) a second lambdoid phage preparation, said preparation comprising second lambdoid phages that display a target-binder molecule,
under conditions permissive for a binding interaction between said target molecule and said target-binder molecule; and
(b) assaying for co-infection of said host cells by a first lambdoid phage and a second lambdoid phage, wherein said co-infection is indicative of a binding interaction between said target molecule and said target-binder-molecule.

2. The method of claim 1, wherein said first lambdoid phages comprise a first genetic mutation that renders the first lambdoid phages incompetent for plaque formation in the absence of a complementary gene in host cells, and wherein said second lambdoid phages comprise a second genetic mutation that renders the second lambdoid phages incompetent for plaque formation in the absence of a complementary gene in host cells, wherein said first lambdoid phages comprise a complementary gene for said second genetic mutation and said second lambdoid phages comprise a complementary gene for said first genetic mutation, and wherein co-infection of said host cells is assayed by assaying for plaque formation.

3. The method of claim 1, wherein at least one of said first lambdoid phage and said second lambdoid phage comprises a display fusion protein comprising said target molecule or said target-binder molecule fused to at least a portion of the lambda gpD protein.

4. The method of claim 3, wherein the target molecule or the target-binder molecule comprises the amino-terminus of the display fusion protein.

5. The method of claim 3, wherein the target molecule or the target-binder molecule comprises the carboxy-terminus of the display fusion protein.

6. The method of claim 1, wherein said first lambdoid phage comprises a display fusion protein comprising said target molecule fused to at least a portion of the lambda gpD protein and said second lambdoid phage comprises a display fusion protein comprising said target-binder molecule fused to at least a portion of the lambda gpD protein.

7. The method of claim 1, wherein at least one of said first lambdoid phage and said second lambdoid phage comprises a display fusion protein comprising said target molecule or said target-binder molecule fused to at least a portion of the lambda gpV protein.

8. The method of claim 1, wherein the first lambdoid phage and the second lambdoid phage are bacteriophage lambda.

9. The method of claim 1, wherein either said first lambdoid phage preparation comprises a library of greater than 10⁶ different target molecules or said second lambdoid phage comprises a library of greater than 10⁶ different target-binder molecules.

10. The method of claim 1, wherein said first lambdoid phage preparation comprises a library of greater than 10⁶ different target molecules, and wherein said second lambdoid phage preparation comprises a library of greater than 10⁶ different target-binder molecules.

11. The method of claim 1, wherein either said first lambdoid phage displays an average of greater than 100 target molecules per phage particle or said second lambdoid phage displays an average of greater than 100 target molecules per phage particle.

12. The method of claim 1, wherein said first lambdoid phage displays an average of greater than 100 target molecules per phage particle, and wherein said second lambdoid phage displays an average of greater than 100 target-binder molecules per phage particle.

13. The method of claim 1, wherein in said step (a) said first lambdoid phage preparation and said second lambdoid phage preparation or combined in a pre-incubation mixture and the pre-incubation mixture is contacted with said host cells.

14. A method for identifying or assessing protein binding modulators comprising the steps of:
(a) incubating host cells under conditions permissive for lambdoid phage infection of said host cells with
(1) a first lambdoid phage preparation, said preparation comprising first lambdoid phages that display a target molecule, and
(2) a second lambdoid phage preparation, said preparation comprising second lambdoid phages that display a target-binder molecule,
under conditions permissive for a binding interaction between said target molecule and said target-binder molecule, in the presence and absence of a test modulator; and
(b) assaying for co-infection of said host cells by a first lambdoid phage and a second lambdoid phage and observing the effect of the test modulator on the number of co-infections, wherein said co-infection is indicative of a binding interaction between said target molecule and said target-binder-molecule, and wherein said test modulator is identified as a protein-binding modulator if the number of co-infections in the presence of the test modulator is greater or less than the number of co-infections in the absence of the test modulator.

15. A method of identifying or assessing a binding interaction between a target molecule and a target-binder molecule comprising the steps of:
(a) mixing
(1) a first lambdoid phage preparation, said preparation comprising first lambdoid phages that display a target molecule, and
(2) a second lambdoid phage preparation, said preparation comprising second lambdoid phages that display a target-binder molecule,
under conditions permissive for a binding interaction between said target molecule and said target-binder molecule; and
(b) assaying for phage complex formation between at least one first lambdoid phage and at least one second lambdoid phage, wherein said phage complex formation is indicative of a binding interaction between said target molecule and said target-binder-molecule.

16. The method of claim 15 wherein at least one of said first lambdoid phages and said second lambdoid phages comprises a marker tag linked to the phage particles.

17. The method of claim 16, wherein the marker tag is a detectable marker tag.

18. The method of claim 16, wherein the marker tag is a ligand marker tag.

19. The method of claim 15, wherein at least one of said first lambdoid phage and said second lambdoid phage comprises a display fusion protein comprising said target molecule or said target-binder molecule fused to at least a portion of the lambda gpD protein.

20. The method of claim 19, wherein the target molecule or the target-binder molecule comprises the amino-terminus of the display fusion protein.

21. The method of claim 19, wherein the target molecule or the target-binder molecule comprises the carboxy-terminus of the display fusion protein.

22. The method of claim 15, wherein said first lambdoid phage comprises a display fusion protein comprising said target molecule fused to at least a portion of the lambda gpD protein and said second lambdoid phage comprises a display fusion protein comprising said target-binder molecule fused to at least a portion of the lambda gpD protein.


SEQUENCE LISTING (5 sequences)

SEQ ID NO: 1 - protein, 5 aa, Artificial
  Synthetic
PGGSG

SEQ ID NO: 2 - protein, 6 aa, Escherichia coli
CCCGGG

SEQ ID NO: 3 - protein, 8 aa, Escherichia coli
GCCCGGGC

SEQ ID NO: 4 - protein, 8 aa, Artificial
  Synthetic
GEGEGEGE

SEQ ID NO: 5 - protein, 8 aa, Artificial
  Synthetic
GRGRGRGR